(19) United States
(12) Patent Application Publication
(10) Pub. No.: US 2024/0091263 A1
(43) Pub. Date: Mar. 21, 2024

(54) CHIMERIC ANTIGEN RECEPTORS TARGETING ALBUMIN AND THEIR METHODS OF USES

(71) Applicant: PORTON ADVANCED SOLUTIONS LTD., Suzhou (CN)

(72) Inventors: Lingjie KONG, Union City, CA (US); Gong CHEN, Suzhou (CN); Jean WANG, Union City, CA (US); Tianwei GUO, Suzhou (CN); Shicheng ZHU, Suzhou (CN)

(21) Appl. No.: 18/479,751

(22) Filed: Oct. 2, 2023

Related U.S. Application Data
(63) Continuation-in-part of application No. PCT/CN2022/085210, filed on Apr. 4, 2022.

(30) Foreign Application Priority Data
Apr. 2, 2021 (WO) PCT/CN2021/085179

(51) Int. Cl. A61K 35/17; A61K 39/00; C07K 14/705; C07K 16/18; C12N 5/0783; C12N 15/86

(52) U.S. Cl. CPC A61K 35/17; A61K 39/4611; A61K 39/4631; A61K 39/464429; C07K 14/70517; C07K 16/18; C12N 5/0636; C12N 15/86; A61K 2239/13; A61K 2239/17; A61K 2239/21; A61K 2239/22; C07K 2317/569; C12N 2740/15043

(57) ABSTRACT

The present disclosure provides a chimeric antigen receptor (CAR) specific for albumin. The present disclosure also provides compositions comprising the CAR, polynucleotides encoding the CAR, vectors comprising a polynucleotide encoding the CAR, engineered cells comprising the CAR, and method using the same.

### CROSS-REFERENCE TO RELATED APPLICATIONS

This application is a continuation-in-part of application No. PCT/CN2022/085210, filed on Apr. 4, 2022, which claims priority PCT application no. PCT/CN2021/085179, filed Apr. 2, 2021, the disclosure of which is incorporated herein by reference.

### SEQUENCE LISTING

The sequence listing that is contained in the file named “077963-8004US01-sequence list”, which is 174,170 bytes and was created on Oct. 2, 2023, is filed herewith by electronic submission and is incorporated by reference herein.

### FIELD OF THE INVENTION

The present disclosure generally relates to the field of cell therapy. In particular, the present disclosure relates to compositions and methods for expanding immune cells and stimulating an immune response in the presence of albumin.

### BACKGROUND

Mammalian immune system is capable of recognizing and eliminating cells that have being infected or damaged as well as those that have become cancerous. In the case of cancer, immune cells such as cytotoxic T cells can bind to the specific antigens on the surface of cancer cells (cancer antigen) and kill the cancer cells. Taking advantage of this natural ability of the immune system, adoptive cell therapies, also known as cellular immunotherapies, have been developed to fight against cancer. Based on the source of the cells and the approach of genetic engineering, cellular immunotherapies can be grouped into tumor-infiltrating lymphocyte (TIL) therapy, engineered T cell receptor (TCR) therapy, chimeric antigen receptor (CAR) T cell therapy and natural killer (NK) cell therapy.

Naturally occurring T cells in cancer patients are often capable of targeting the cancer cells. The existence of these T cells alone, however, isn't always enough to guarantee that they will be able to eliminate tumors. One potential impediment is that these T cells must first be activated, and then maintain the activity for a sufficiently long time to sustain an effective anti-tumor response. To address these issues, TIL therapy harvests naturally occurring T cells that have already infiltrated patients' tumors, and then activates and expands them. Large numbers of these activated T cells are then re-infused into patients to destroy tumors.

One problem with the TIL therapy is that not all patients have T cells that have already recognized their tumors. To solve this issue, TCR therapy takes T cells from patients and equips the T cells with a new T cell receptor that enables the T cells to target specific cancer antigens.

Both TIL and TCR therapies can only target and eliminate cancer cells that present their antigens via major histocompatibility complex (MHC). To overcome this limit, scientists have engineered T cells or NK cells to express CAR that specifically binds to the antigens expressed on the surface of cancer cells even if these antigens are not presented on the surface via MHC.

Current cellular immunotherapies involve a step of activating and/or expanding the immune cells isolated from human subject, which is costly and time consuming. As a result, ex vivo activation and/or expansion of the immune cells has become one of the major obstacles that prevent cellular immunotherapies from wide implementation. Therefore, a need exists for developing new methods for activating and/or expanding immune cells for cellular immunotherapies.

### SUMMARY OF INVENTION

In one aspect, the present disclosure provides a polynucleotide encoding a chimeric antigen receptor (CAR). In some embodiments, the CAR comprising (1) an extracellular domain comprising a first antigen-binding domain, (2) a transmembrane domain and (3) an intracellular signaling domain, wherein the first antigen binding domain specifically binds to albumin.

In some embodiments, the first antigen-binding domain is a single-chain variable fragment (scFv). In some embodiments, the scFv comprises a variable heavy (VH) and variable light (VL) region. In some embodiments, the VH comprises an HCDR1 having a sequence as shown in Table 1, or a sequence having at least 90% identity thereto, or a sequence having 1, 2, 3 amino acid residue difference therefrom; an HCDR2 having a sequence as shown in Table 2, or a sequence having at least 90% identity thereto, or a sequence having 1, 2, 3 amino acid residue difference therefrom; and an HCDR3 having a sequence as shown in Table 3, or a sequence having at least 90% identity thereto, or a sequence having 1, 2, 3 amino acid residue difference therefrom. In some embodiments, the VL region comprises a LCDR1 having a sequence as shown in Table 4, or a sequence having at least 90% identity thereto, or a sequence having 1, 2, 3 amino acid residue difference therefrom; a LCDR2 having a sequence as shown in Table 5, or a sequence having at least 90% identity thereto, or a sequence having 1, 2, 3 amino acid residue difference therefrom; and a LCDR3 having a sequence as shown in Table 6, or a sequence having at least 90% identity thereto, or a sequence having 1, 2, 3 amino acid residue difference therefrom.

In some embodiments, the antigen-binding domain is a single domain antibody (SDAB). In some embodiments, the SDAB comprises a CDR1 having a sequence as shown in Table 1, or a sequence having at least 90% identity thereto, or a sequence having 1, 2, 3 amino acid residue difference therefrom; a CDR2 having a sequence as shown in Table 2, or a sequence having at least 90% identity thereto, or a sequence having 1, 2, 3 amino acid residue difference therefrom; and a CDR3 having a sequence as shown in Table 3, or a sequence having at least 90% identity thereto, or a sequence having 1, 2, 3 amino acid residue difference therefrom.

In some embodiments, the antigen-binding domain is a nanobody. In some embodiments, the nanobody comprises a CDR1 having a sequence as shown in Table 3, or a sequence having at least 90% identity thereto, or a sequence having 1, 2, 3 amino acid residue difference therefrom; a CDR2 having a sequence as shown in Table 3, or a sequence having at least 90% identity thereto, or a sequence having 1, 2, 3 amino acid residue difference therefrom; and a CDR3 having a sequence as shown in Table 3, or a sequence having at least 90% identity thereto, or a sequence having 1, 2, 3 amino acid residue difference therefrom.

In some embodiments, the CAR further comprises a signal peptide. In some embodiments, the signal peptide comprises a signal peptide of CD8 alpha. In some embodiments, the signal peptide of CD8 alpha comprises the sequence of SEQ ID NO: 130 or a sequence having at least 90% identity thereto; or a sequence having 1, 2, 3 amino acid residue difference therefrom.

In some embodiments, the transmembrane domain comprises a transmembrane domain of CD8 alpha. In some embodiments, the transmembrane domain of CD8 alpha comprises the sequence of SEQ ID NO: 132, or a sequence having at least 90% identity thereto; or a sequence having 1, 2, 3 amino acid residue difference therefrom.

In some embodiments, the extracellular domain is linked to the transmembrane domain by a hinge region. In some embodiments, the hinge region comprises a hinge region of CD8 alpha. In some embodiments, the hinge region of CD8 alpha comprises the sequence of SEQ ID NO: 133, or a sequence having at least 90% identity thereto; or a sequence having 1, 2, 3 amino acid residue difference therefrom.

In some embodiments, the intracellular domain comprises a costimulatory domain and a signaling domain. In some embodiments, the costimulatory domain comprises an intracellular domain of CD137. In some embodiments, the intracellular domain of CD137 comprises the sequence of SEQ ID NO: 134, or a sequence having at least 90% identity thereto; or a sequence having 1, 2, 3 amino acid residue difference therefrom.

In some embodiments, the intracellular domain comprises a signaling domain of CD3 zeta. In some embodiments, the signaling domain of CD3 zeta comprises the sequence of SEQ ID NO: 135, or a sequence having at least 90% identity thereto

In some embodiments, the CAR has the structure: S-AB-H-TM-IC wherein S is a signal peptide, AB is the antigen-binding domain, H is a hinge region, TM is the transmembrane domain and IC is the intracellular signaling domain. In some embodiments, the CAR has the structure: S-VH-L-VL-H-TM-IC, wherein VH is a variable heavy region, L is a linker, VL is a variable light region. In some embodiments, the CAR has the structure: S-VL-L-VH-H-TM-IC, wherein VH is a variable heavy region, L is a linker, VL is a variable light region.

In some embodiments, the CAR has the structure: S-SDAB-TM-IC, wherein SDAB is a single domain antibody.

In some embodiments, the CAR has the structure: S-N-TM-IC, wherein N is a nanobody.

In some embodiments, the extracellular domain further comprises a second antigen-binding domain specifically binding to a different epitope of albumin from the first antigen-binding domain.

In some embodiments, the extracellular domain further comprises a second antigen-binding domain specifically binding to a cell surface antigen. In some embodiments the cell surface antigen is a cancer antigen. In some embodiments, the cancer antigen is selected from the group consisting of CD19, CD20, CAIX, CD33, CD44v7/8, CEA, EGP-2, EGP-40, erb-B2, erb-B3, erb-B4, FBP, fetal acetyl choline receptor, GD2, GD3, Her2/neu, IL-13R-a2, KDR, k-light chain, LeY, LI cell adhesion molecule, MAGE-A1, mesothelin, MUCI, KG2D ligands, oncofetal antigen (h5T4), PSCA, PSMA, TAA, TAG-72, and VEGF-R.

In some embodiments, the extracellular domain further comprising a second antigen-binding domain specifically binding to albumin, wherein the first and second antigen-binding domains bind different epitopes of albumin. In some embodiments, the extracellular domain further comprises a third antigen-binding domain specifically binding to a cancer antigen.

In some embodiments, the polynucleotide provided herein is a DNA. In some embodiments, the polynucleotide provided herein is an RNA.

In another aspect, the present disclosure provides a polypeptide encoded by the polynucleotide provided herein.

In another aspect, the present disclosure provides a vector comprising the polynucleotide provided herein, wherein the polynucleotide encoding the CAR is operatively linked to at least one regulatory polynucleotide element for expression of the CAR.

In some embodiments, the vector is a plasmid vector, a viral vector, a transposon, a site directed insertion vector, or a suicide expression vector. In some embodiments, the vector is a lentiviral vector, a retroviral vector, or an AAV vector.

In another aspect, the present disclosure provides an engineered cell comprising the polynucleotide provided herein. In some embodiments, the engineered cell is a T cell or an NK cell.

In some embodiments, the engineered cell further comprises a second CAR, wherein the second CAR comprises a second antigen-binding domain specifically binding to a cancer antigen. In some embodiments, the cancer antigen is selected from the group consisting of CD19, CD20, CAIX, CD33, CD44v7/8, CEA, EGP-2, EGP-40, erb-B2, erb-B3, erb-B4, FBP, fetal acetyl choline receptor, GD2, GD3, Her2/neu, IL-13R-a2, KDR, k-light chain, LeY, LI cell adhesion molecule, MAGE-A1, mesothelin, MUC, KG2D ligands, oncofetal antigen (h5T4), PSCA, PSMA, TAA, TAG-72, and VEGF-R.

In some embodiments, the engineered cell further comprises a second CAR, wherein the second CAR comprises a second antigen-binding domain specifically binding to albumin, wherein the first and second antigen-binding domains bind different epitopes of albumin. In some embodiments, the engineered cell further comprises a third CAR, wherein the third CAR comprises a third antigen-binding domain specifically binding to a cancer antigen.

In another aspect, the present disclosure provides a method for stimulating an immune response comprising contacting the engineered cell provided herein with albumin. In some embodiments, the engineered cell is contacted with the albumin ex vivo. In some embodiments, the engineered cell is contacted with the albumin in vivo by administering the engineered cell to a subject in need of immune stimulation.

In some embodiments, the subject has cancer. In some embodiments, the cancer is a solid cancer selected from the group consisting of adrenal cancer, bone cancer, brain cancer, breast cancer, colorectal cancer, esophageal cancer, eye cancer, gastric cancer, head and neck cancer, kidney cancer, liver cancer, lung cancer, non-small cell lung cancer, bronchioloalveolar cell lung cancer, mesothelioma, head and neck cancer, squamous cell carcinoma, melanoma, oral cancer, ovarian cancer, cervical cancer, penile cancer, prostate cancer, pancreatic cancer, skin cancer, sarcoma, testicular cancer, thyroid cancer, uterine cancer, vaginal cancer. In some embodiments, the cancer is a hematologic malignancy selected from the group consisting of acute lymphocytic leukemia (ALL), acute myeloid leukemia (AML), B-cell leukemia, chronic lymphoblastic leukemia (CLL), blastic plasmacytoid dendritic cell neoplasm (BPDCN), chronic myelomonocytic leukemia (CMML), chronic myelocytic leukemia (CML), pre-B acute lymphocytic leukemia (Pre-B ALL), diffuse large B-cell lymphoma (DLBCL), extranodal NK/T-cell lymphoma, hairy cell leukemia, heavy chain disease, HHV8-associated primary effusion lymphoma, plasmablastic lymphoma, primary CNS lymphoma, primary mediastinal large B-cell lymphoma, T-cell/histiocyte-rich B-cell lymphoma, Hodgkin's lymphoma, non-Hodgkin's lymphoma, Waldenstrom's macroglobulinemia, multiple myeloma (MM), myelodysplastic syndromes (MDS), myeloproliferative neoplasms, and polycythemia vera.

In some embodiments, stimulating an immune response comprises increasing expression and/or secretion of immune stimulating cytokines and/or molecules. In some embodiments, the immune stimulating cytokines and/or molecules are one or more of TNF-a, IFN-β, IFN-γ, IL-1, IL-2, IL-4, IL-6, IL-8, IL-10, IL-12, IL-18 and granulocyte-macrophage colony stimulating factor.

In some embodiments, stimulating an immune response comprises increasing proliferation of immune cells. In some embodiments, the immune cells are T cells.

In another aspect, the present disclosure provides a method for expanding cells in vitro, the method comprising contacting the engineered cell provided herein in vitro with a composition comprising albumin. In some embodiments, the composition further comprises IL-2. In some embodiments, the method further comprises contacting the engineered cell with feeder cells. In some embodiments, the feeder cells are irradiated.

In another aspect, the present disclosure provides a method for treating a disease or pathological condition in a patient comprising administering a therapeutically effective amount of the engineered cell provided herein to the patient. In some embodiments, the disease is cancer.

In some embodiments, the method for treating a disease or pathological condition further comprises expanding the engineered cells in vitro by a method comprising contacting the engineered cell in vitro with a composition comprising albumin.

### BRIEF DESCRIPTION OF THE DRAWINGS

The accompanying drawings, which are incorporated herein, form part of the specification. Together with this written description, the drawings further serve to explain the principles of, and to enable a person skilled in the relevant art(s), to make and use the present disclosure.

FIG. 1A illustrates the activation of immune cell by a CAR for albumin. An albumin CAR is introduced into immune cells. An albumin dimer induces CAR dimerization and activation (shaded) of the engineered immune cells.

FIG. 1B illustrates the activation of immune cell by two CARs for albumin. The first CAR comprises a first antigen-binding domain and the second CAR comprises a 2nd antigen-binding domain to albumin. Both CARs are expressed in the same engineered immune cells, and bind to separate epitopes of the same albumin molecule. When the two CARs dimerize through the same albumin molecule, the engineered immune cells are activated.

FIG. 1C illustrates the activation of immune cell by a CAR comprising two albumin binding domains. When the CAR is expressed in engineered immune cells, one albumin molecule binds two CAR molecules to bridge the CAR dimerization and the cell activation.

FIG. 1D illustrate transient expression of CAR targeting albumin. CARs are expressed through DNA or mRNA transfection, cells are activated by albumin. Activated cells proliferate, and the final cells do not have albumin CARs.

FIG. 2 illustrates a schematic diagram of an exemplary CAR construct.

FIG. 3 illustrates that expressing of the CAR against albumin enhances T cell expansion ex vivo. An albumin CAR was delivered into T cells through lentivirus vector. Cell growth was monitored by cell counting. Compared to the control cells, which expanded over 200 times in around 30 days, the CAR positive cells expanded over 60,000 times.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definition

The following definitions are provided to assist the reader. Unless otherwise defined, all terms of art, notations and other scientific or medical terms or terminology used herein are intended to have the meanings commonly understood by those of skill in the art. In some cases, terms with commonly understood meanings are defined herein for clarity and/or for ready reference, and the inclusion of such definitions herein should not necessarily be construed to represent a substantial difference over the definition of the term as generally understood in the art.

As used herein, the singular forms “a”, “an” and “the” include plural references unless the context clearly dictates otherwise.

“Antigen” refers to a molecule that provokes an immune response. This immune response may be either humoral, or cell-mediated response, or both. The skilled artisan will understand that any macromolecule, including virtually all proteins or peptides, can serve as an antigen. It is readily apparent that the present disclosure includes therapeutic antibodies acting as antigen eliciting immune response.

“Antibody” refers to a polypeptide of the immunoglobulin (Ig) family that binds with an antigen. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR) (light chain CDRs including LCDR1, LCDR2, and LCDR3, heavy chain CDRs including HCDR1, HCDR2, HCDR3), interspersed with regions that are more conserved, termed framework regions (FR). CDR boundaries for the antibodies disclosed herein may be defined or identified by the conventions of Kabat, IMGT, Chothia, or AI-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et al., National Institutes of Health, Bethesda, Md. (1991); Marie-Paule Lefranc et al, Developmental and Comparative Immunology, 27: 55-77 (2003); Marie-Paule Lefranc et al, Immunome Research, 1(3), (2005); Marie-Paule Lefranc, Molecular Biology of B cells (second edition), chapter 26, 481-514, (2015)). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen.

“Antigen-binding fragment” as used herein refers to an antibody fragment formed from a portion of an intact antibody comprising one or more CDRs, or any other antibody fragment that can bind to an antigen but does not comprise an intact native antibody structure. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), single-chain Fv-Fc antibody (scFv-Fc), an scFv dimer (bivalent diabody), a bispecific antibody, a multispecific antibody, a camelized single domain antibody, a nanobody, a domain antibody, and a bivalent domain antibody. An antigen-binding fragment is capable of binding to the same antigen to which the parent antibody binds.

“Autologous” cells refer to any cells derived from the same subject into which they are later to be re-introduced.

“Allogeneic” cells refer to any cells derived from a different subject of the same species.

“Co-stimulatory ligand” refers to a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an major histocompatibility complex (MHC) molecule loaded with peptide, mediates a T cell response, including, but not limited to, proliferation, activation, differentiation, and the like.

“Co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

“Effector cells” used in the context of immune cells refers to cells that can be activated to carry out effector functions in response to stimulation. Effector cells may include, without limitation, NK cells, cytotoxic T cells and helper T cells.

“Effective amount” or “therapeutically effective amount” refers to an amount of a cell, composition, formulation or any material as described here effective to achieve a desirable biological result. Such results may include, without limitation, elimination of B cells expressing a specific BCR and the antibodies produced therefrom.

“Epitope” refers to a portion of an antigen recognized by an antibody or an antigen-binding fragment thereof. An epitope can be linear or conformational.

Percentage of “identity” or “sequence identity” in the context of polypeptide or polynucleotide is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

“Operatively linked” refers to a functional relationship between two or more polynucleotide sequences. In the context of a polynucleotide encoding a fusion protein, such as a polypeptide chain of a CAR of the disclosure, the term means that the two or more polynucleotide sequences are joined such that the amino acid sequences encoded by these segments remain in-frame. In the context of transcriptional or translational regulation, the term refers to the functional relationship of a regulatory sequence to a coding sequence, for example, a promoter in the correct location and orientation to the coding sequence so as to modulate the transcription.

“Polynucleotide” or “nucleic acid” refers to a chain of nucleotides. As used herein polynucleotides include all polynucleotide sequences which are obtained by any means available in the art, including, without limitation, recombinant means by synthetic means.

“Polypeptide,” and “protein” are used interchangeably, and refer to a chain of amino acid residues covalently linked by peptide bonds. The polypeptides include natural peptides, recombinant peptides, synthetic peptides, or a combination thereof.

“Single chain variable fragment” or “single-chain Fv antibody” or “scFv” refers to an engineered antibody comprises a light chain variable region fused to a heavy chain variable region directly or via a peptide linker sequence.

“T cell receptor” or “TCR” refers to a protein complex on the surface of T cells that is responsible for recognizing fragments of antigen as peptides bound to MHC molecules.

“Vector” refers to a vehicle into which a polynucleotide may be operably inserted so as to deliver, replicate or express the polynucleotide. A vector may contain a variety of regulatory elements including, without limitation, origin of replication, promoter, transcription initiation sequences, enhancer, selectable marker genes, and reporter genes. A vector may also include materials to aid in its entry into a host cell, including but not limited to a viral particle, a liposome, or ionic or amphiphilic compounds.

It is noted that in this disclosure, terms such as “comprises”, “comprised”, “comprising”, “contains”, “containing” and the like have the meaning attributed in United States Patent law; they are inclusive or open-ended and do not exclude additional, un-recited elements or method steps. Terms such as “consisting essentially of” and “consists essentially of” have the meaning attributed in United States Patent law; they allow for the inclusion of additional ingredients or steps that do not materially affect the basic and novel characteristics of the claimed invention. The terms “consists of” and “consisting of” have the meaning ascribed to them in United States Patent law; namely that these terms are close ended.

### Chimeric Antigen Receptor

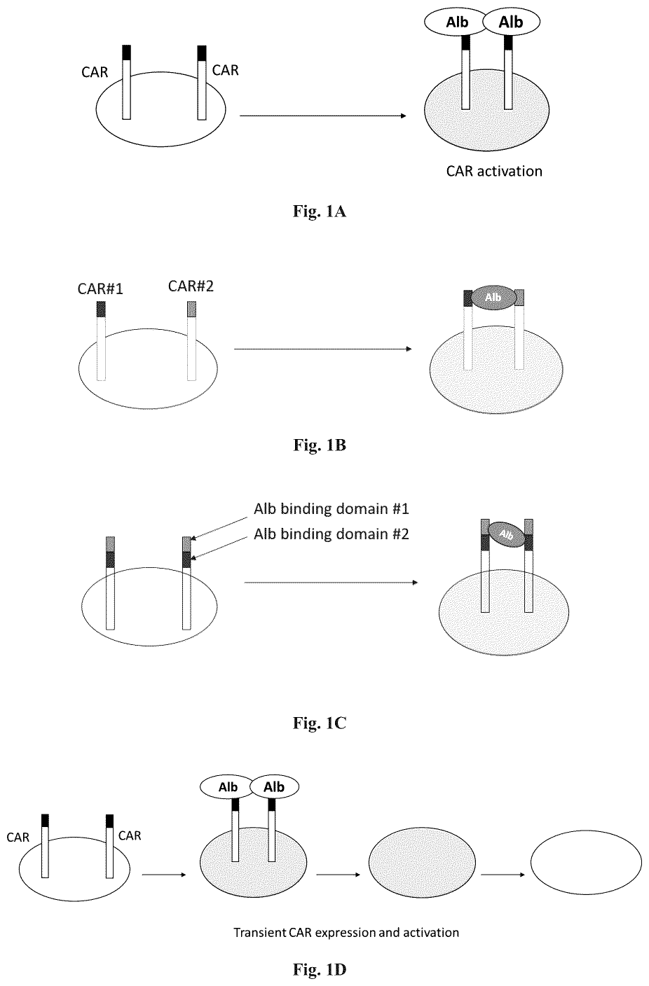

Current cellular immunotherapies involve a step of activating and/or expanding the immune cells isolated from human subject, which is costly and time consuming. Ex vivo activation and/or expansion of the immune cells is one of the major obstacles that prevent cellular immunotherapies from wide implementation. The present disclosure in one aspect relates to a chimeric antigen receptor (CAR) that specifically recognize albumin. Soluble form of antigen usually lacks the ability to trigger CAR signaling as antigen-induced dimerization of CAR is required for CAR activation. Albumin is quite soluble, but frequent encounters between albumin molecules provide an opportunity to form well-defined aggregates: dimers, trimers, or even larger structures. Without wishing to be bound by any theory, the CAR disclosed herein is able to response to an albumin aggregate, thereby activating the immune cell harboring the CAR (FIG. 1A). The CAR disclosed herein is useful in enhancing immune cell (e.g., T cell) activation and expansion in response to albumin ex vivo, in vitro or in vivo. The CAR can also be used as an accessory receptor to boost T-cell mediated responses in all adoptive T-cell therapies, whether based on tumor infiltrating lymphocytes, T-cell receptor (TCR) engineering, or other CARs.

In one aspect, the present disclosure provides a CAR comprising an extracellular domain specifically binding to albumin, a transmembrane domain and an intracellular signaling domain. In another aspect, the present disclosure provides a polynucleotide encoding the CAR described herein.

The term “albumin” or “serum albumin” refers to an albumin (a type of globular protein) found in vertebrate blood. Serum albumin is produced by liver, occurs dissolved in blood plasma and is the most abundant blood protein in mammals. In some embodiments, the serum albumin is selected from human serum albumin (HSA), cynomolgus monkey serum albumin and mouse serum albumin. In some embodiments, the serum albumin provided herein is HSA.

Serum albumin is highly soluble and can aggregate into a dimer, a trimer or even larger structures. In some embodiments, the albumin forms a covalently linked dimer, for example, a dimer with a disulfide bound linking Cys-34. In some embodiments, the albumin form non-covalent dimers with a well-defined structure. The formation of non-covalent dimer can occur at physiologically relevant concentrations or in response to changes in conditions of, e.g., pH, hydrodynamics or temperatures. The non-covalent dimer formation can be readily reversible into monomers. Dimerization is relevant to the role of HSA in the transport, binding, and other physiological processes (Chubarov A., et al., Molecules 2021, 26, 108).

### Extracellular Domain

In some embodiments, the extracellular domain of the CAR comprises a first antigen-binding domain that specifically binds albumin. The first antigen-binding domain can be an antibody or an antigen-binding fragment thereof (e.g., a Fv, a Fab, a (Fab)2, an scFv, a SDAB, a nanobody), or any alternative scaffold known in the art to function as antigen binding domain.

In some embodiments, the first antigen-binding domain is derived from the same species in which the CAR will ultimately be used in. For example, it may be beneficial for the antigen binding domain of the CAR to comprise human or humanized residues for the antigen binding domain of an antibody or antibody fragment. In some embodiments, the antigen binding domain comprises a human or humanized antibody or an antibody fragment thereof. The term “human antibody” refers to an antibody where the whole molecule is of human origin or consists of an amino acid sequence identical to a human form of the antibody or immunoglobulin. The term “humanized antibody” refers to an antibody which contains sequence (e.g., CDR sequences) derived from non-human immunoglobulin. Human or humanized antibodies or fragments thereof may be prepared in a variety of ways, for example through recombinant methodologies or through immunization with an antigen of interest of a mouse that is genetically modified to express antibodies derived from human heavy and/or light chain-encoding genes.

In some embodiments, the first antigen-binding domain is a single-chain variable fragment (scFv). An scFv can comprise a peptide linker of at least 1, 2, 3, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. Variation in the linker length may retain or enhance activity.

In some embodiments, the scFv comprises a variable heavy (VH) and variable light (VL) region. In some embodiments, the VH comprises a CDR1 having a sequence as shown in Table 1, or a sequence having at least 90% identity thereto, or a sequence having 1, 2, 3 amino acid residue difference therefrom; a CDR2 having a sequence as shown in Table 2, or a sequence having at least 90% identity thereto, or a sequence having 1, 2, 3 amino acid residue difference therefrom; and a CDR3 having a sequence as shown in Table 3, or a sequence having at least 90% identity thereto, or a sequence having 1, 2, 3 amino acid residue difference therefrom. In some embodiments, the VL region comprises a CDR1 having a sequence as shown in Table 4, or a sequence having at least 90% identity thereto, or a sequence having 1, 2, 3 amino acid residue difference therefrom; a CDR2 having a sequence as shown in Table 5, or a sequence having at least 90% identity thereto, or a sequence having 1, 2, 3 amino acid residue difference therefrom; and a CDR3 having a sequence as shown in Table 6, or a sequence having at least 90% identity thereto, or a sequence having 1, 2, 3 amino acid residue difference therefrom.

TABLE l

| CDR1 | Clone No. | Sequence | SEQ ID NO. |
|---|---|---|---|
| HCDR1 | Clone 1 | NYAIN | 1 |
| HCDR1 | Clone 2 | NYGIH | 2 |
| HCDR1 | Clone 3 | GFSLSSNAMG | 3 |
| HCDR1 | Clone 4 | GFSFSSSYWIC | 4 |

TABLE 2

| CDR2 | Clone No. | Sequence | SEQ ID NO. |
|---|---|---|---|
| HCDR2 | Clone 1 | IIWASGTTFYATWAKG | 5 |
| HCDR2 | Clone 2 | SISPSGGLTYYRDSVKG | 6 |
| HCDR2 | Clone 3 | IISVGGFTYYASWAKG | 7 |
| HCDR2 | Clone 4 | CVFTGDGTTYYASWAKG | 8 |

TABLE 3

| CDR3 | Clone No. | Sequence | SEQ ID NO. |
|---|---|---|---|
| HCDR3 | Clone 1 | TVPGYSTAPYFDL | 9 |
| HCDR3 | Clone 2 | GGEGIFDY | 10 |
| HCDR3 | Clone 3 | RDRHGGDSSGAFLY | 11 |
| HCDR3 | Clone 4 | RPVSVYYYGMDL | 12 |

TABLE 4

| CDR1 | Clone No. | Sequence | SEQ ID NO. |
|---|---|---|---|
| LCDR1 | Clone 1 | QSSPSVWSNFLS | 13 |
| LCDR1 | Clone 2 | CRATQSIYNALA | 14 |
| LCDR1 | Clone 3 | QSSESVYSNNQLS | 15 |
| LCDR1 | Clone 4 | QASQIISSRSA | 16 |

TABLE 5

| CDR2 | Clone No. | Sequence | SEQ ID NO. |
|---|---|---|---|
| LCDR2 | Clone 1 | EASKLTS | 17 |
| LCDR2 | Clone 2 | NANTLHT | 18 |
| LCDR2 | Clone 3 | DASDLAS | 19 |
| LCDR2 | Clone 4 | QASKLAS | 20 |

TABLE 6

| CDR3 | Clone No. | Sequence | SEQ ID NO. |
|---|---|---|---|
| LCDR3 | Clone 1 | GGGYSSISDTT | 21 |
| LCDR3 | Clone 2 | QQYYDYPLT | 22 |
| LCDR3 | Clone 3 | AGGFSSSSDTA | 23 |
| LCDR3 | Clone 4 | QCTYIDSNFGA | 24 |

In some embodiments, the VH comprises CDR1, CDR2 and CDR3 having sequences selected from the group consisting of: (1) SEQ ID NOs: 1, 5 and 9, (2) SEQ ID NOs: 2, 6 and 10, (3) SEQ ID NOs: 3, 7 and 11, and (4) SEQ ID NOs: 4, 8 and 12, or a sequence having at least 90% identity thereto, or a sequence having 1, 2, 3 amino acid residue difference therefrom. In some embodiments, the VL comprises a set of CDR1, CDR2 and CDR3 having sequences selected from the group consisting of: (1) SEQ ID NOs: 13, 17 and 21, (2) SEQ ID NOs: 14, 18 and 22, (3) SEQ ID NOs: 15, 19 and 23, and (4) SEQ ID NOs: 16, 19 and 24, or a sequence having at least 90% identity thereto, or a sequence having 1, 2, 3 amino acid residue difference therefrom.

In some embodiments, the scFv comprises:
a VH comprising SEQ ID NOs: 1, 5 and 9, and a VL comprising SEQ ID NOs: 13, 17 and 21;
a VH comprising SEQ ID NOs: 2, 6 and 10, and a VL comprising SEQ ID NOs: 14, 18 and 22;
a VH comprising SEQ ID NOs: 3, 7 and 11, and a VL comprising SEQ ID NOs: 15, 19 and 23; or
a VH comprising SEQ ID NOs: 4, 8 and 12, and a VL comprising SEQ ID NOs: 16, 20 and 24.

In some embodiments, the scFv comprises a VH and a VL comprising the sequences listed in Table 7, or sequences having at least 80% (e.g. at least 85%, 88%, 90%, 91%, 92%, 93%, 94%, 95%, 96%, 97%, 98%, or 99% No) identity thereto or sequences having 1, 2, 3, 6, 7, 8, 9, 10 amino acid residue difference therefrom. In some embodiments, the difference occur in regions outside the CDRs (e.g., in the FRs).

TABLE 7

| | |
|---|---|
| Clone 1 | VH (SEQ ID NO: 25) |
| | EVQLLESGGGLVQPGGSLRLSCAVSGIDLSNYAINWVRQAPGKCLEWIGII |
| | WASGTTFYATWAKGRFTISRDSTTVYLQMNSLRAEDTAVYYCARTVPGY |
| | STAPYFDLWGQGTLVTVSS |
| | VL (SEQ ID NO: 26) |
| | DIVMTQSPSSVSASVGDRVTITCQSSPSVWSNFLSWYQQKPGKAPKLLIYE |
| | ASKLTSGVPSRFKGSGSGTDFTLTISSLQPEDFATYYCGGGYSSISDTTFGC |
| | GTKVEIK |
| Clone 2 | VH (SEQ ID NO: 27) |
| | EVQLVESGGGLVQPGGSLRLSCAASGFSLSSNAMGWVRQAPGKGLEYIGI |
| | ISVGGFTYYASWAKGRFTISRDNSKNTVYLQMNSLRAEDTATYFCARDR |
| | HGGDSSGAFYLWGQGTLVTVSS |
| | VL (SEQ ID NO: 28) |
| | DIQMTQSPSSLSASVGDRVTITCQSSESVYSNNQLSWYQQKPGQPPKLLIY |
| | DASDLASGVPSRFSGSGSGTDFTLTISSLQPEDFATYYCAGGFSSSSDTAFG |
| | GGTKLTVLG |

In some embodiments, the scFv comprises a sequence listed in Table 8, or sequences having at least 80% (e.g., at least 85%, 88%, 90%, 91%, 92%, 93%, 94%, 95%, 96%, 97%, 98%, or 99%) identity thereto or sequences having 1, 2, 3, 6, 7, 8, 9, 10 amino acid residue difference therefrom. In some embodiments, the difference occur in regions outside the CDRs (e.g., in the FRs).

TABLE 8

| | |
|---|---|
| scFv 1 | scFv (SEQ ID NO: 29) |
| | EVQLLESGGGLVQPGGSLRLSCAVSGIDLSNYAINWVRQAPGKCLEWIGII |
| | WASGTTFYATWAKGRFTISRDSTTVYLQMNSLRAEDTAVYYCARTVPGY |
| | STAPYFDLWGQGTLVTVSSGGGGSDIVMTQSPSSVSASVGDRVTITCQSS |
| | PSVWSNFLSWYQQKPGKAPKLLIYEASKLTSGVPSRFKGSGSGTDFTLTIS |
| | SLQPEDFATYYCGGGYSSISDTTFGCGTKVEIK |
| scFv 2 | scFv (SEQ ID NO: 30) |
| | EVQLVESGGGLVQPGGSLRLSCAASGFSLSSNAMGWVRQAPGKGLEYIGI |
| | ISVGGFTYYASWAKGRFTISRDNSKNTVYLQMNSLRAEDTATYFCARDR |
| | HGGDSSGAFYLWGQGTLVTVSSGGGGSDIQMTQSPSSLSASVGDRVTITC |
| | QSSESVYSNNQLSWYQQKPGQPPKLLIYDASDLASGVPSRFSGSGSGTDF |
| | TLTISSLQPEDFATYYCAGGFSSSSDTAFGGGTKLTVLG |

In some embodiments, the first antigen-binding domain is a single domain antibody (SDAB). The term “single domain antibody” refers to an antibody fragment containing a single variable domain of a heavy chain or a single variable domain of a light chain. In some embodiments, a single domain antibody contains 1, 2, or 3 complementarity determining regions (CDRs). A single domain antibody is fairly small in size, for example, has a molecular weight of no more than 25 kD, no more than 20 kD, no more than 15 kD, or no more than 10 kD. In some embodiments, a single domain antibody is a human antibody or humanized antibody.

In some embodiments, the single variable domain is derived from the variable domain of a conventional antibody (e.g., from humans or mice) heavy chain (VH domain) or the variable domain of a common antibody light chain (VL domain).

In some embodiments, the SDAB comprises a CDR1 having a sequence as shown in Table 9, or a sequence having at least 90% identity thereto, or a sequence having 1, 2, 3 amino acid residue difference therefrom; a CDR2 having a sequence as shown in Table 10, or a sequence having at least 90⅙ identity thereto, or a sequence having 1, 2, 3 amino acid residue difference therefrom, and a CDR3 having a sequence as shown in Table 11, or a sequence having at least 90% identity thereto, or a sequence having 1, 2, 3 amino acid residue difference therefrom.

TABLE 9

| | | | SEQ ID |
|---|---|---|---|
| CDR | Clone # | Sequence | NO. |
| LCDR1 | dAb7m12 | RASQSIFRHLK | 31 |
| LCDR1 | dAb7m16 | RASQSIIKHLK | 32 |
| LCDR1 | dAb7m26 | RASQSIYYHLK | 33 |
| LCDR1 | dAb7r1 | RASQYIGRYLR | 34 |
| LCDR1 | dAb7r3 | RASQYIGRYLR | 34 |
| LCDR1 | dAb7r4 | RASQWIGRYLR | 35 |
| LCDR1 | dAb7r7 | RASQYIGRYLR | 34 |
| LCDR1 | dAb7r5 | RASQYISRQLR | 36 |
| LCDR1 | dAb7r8 | RASQWIHRQLK | 37 |
| LCDR1 | dAb7r13 | RASQHIHRELR | 38 |
| LCDR1 | dAb7r14 | RASQHIHRELR | 38 |
| LCDR1 | dAb7r15 | RASQSIGRRLK | 39 |
| LCDR1 | dAb7r16 | RASQKIYKNLR | 40 |
| LCDR1 | dAb7r17 | RASQKIYNNLR | 41 |
| LCDR1 | dAb7r18 | RASQWIYKSLG | 42 |
| LCDR1 | dAb7r19 | RASQWIYRHLR | 43 |
| LCDR1 | dAb7h1 | RASQSISSYLN | 44 |
| LCDR1 | dAb7h6 | RASQSISSYLN | 44 |
| LCDR1 | dAb7h7 | RASQSISSYLN | 44 |
| LCDR1 | dAb7h8 | RASQSISSYLN | 44 |
| LCDRI | dAb7h2 | RASQKIATYLN | 45 |
| HCDR1 | dAb7r20 | PYTMS | 46 |
| HCDR1 | dAb7r21 | PYTMS | 46 |
| HCDR1 | dAb7r22 | PYTMS | 46 |
| HCDR1 | dAb7r23 | PYTMS | 46 |
| HCDR1 | dAb7r24 | PYTMS | 46 |
| HCDR1 | dAb7r25 | PYTMS | 46 |
| HCDR1 | dAb7r26 | PYTMS | 46 |
| HCDR1 | dAb7r27 | PYTMS | 46 |
| HCDR1 | dAb7r28 | AYQMA | 47 |
| HCDR1 | dAb7r29 | DYDMT | 48 |
| HCDR1 | dAb7r30 | DYVMG | 49 |
| HCDR1 | dAb7r31 | HYRMG | 50 |
| HCDR1 | dAb7r32 | WDKMG | 51 |
| HCDR1 | dAb7r33 | AYPMS | 52 |
| HCDR1 | dAb7h30 | TYTMA | 53 |
| HCDR1 | dAb7h31 | PTNMS | 54 |

TABLE 10

| | | | SEQ ID |
|---|---|---|---|
| CDR | Clone # | Sequence | NO. |
| LCDR2 | dAb7m12 | AASRLQS | 55 |
| LCDR2 | dAb7m16 | GASRLQS | 56 |
| LCDR2 | dAb7m26 | KASTLQS | 57 |
| LCDR2 | dAb7rl | DSSVLQS | 58 |
| LCDR2 | dAb7r3 | DSSVLQS | 58 |
| LCDR2 | dAb7r7 | DSSVLQS | 58 |
| LCDR2 | dAb7r4 | NGSQLQS | 59 |
| LCDR2 | dAb7r5 | GAVSLQS | 60 |
| LCDR2 | dAb7r8 | YASILQS | 61 |
| LCDR2 | dAb7r13 | QASRLQS | 62 |
| LCDR2 | dAb7r14 | QASRLQS | 62 |
| LCDR2 | dAb7r15 | RTSWLQS | 63 |
| LCDR2 | dAb7r16 | NSSILQS | 64 |
| LCDR2 | dAb7r17 | NTSILQS | 65 |
| LCDR2 | dAb7r18 | QSSLLQS | 66 |
| LCDR2 | dAb7r19 | DASRLQS | 67 |
| LCDR2 | dAb7h1 | RNSFLQS | 68 |
| LCDR2 | dAb7h2 | RSSSLQS | 69 |
| LCDR2 | dAb7h6 | RLSVLQS | 70 |
| LCDR2 | dAb7h7 | RNSQLQS | 71 |
| LCDR2 | dAb7h8 | RNSPLQS | 72 |
| HCDR2 | dAb7r20 | TISPFGSTTYYADSVKG | 73 |
| HCDR2 | dAb7r21 | TISPFGSTTYYADSVKG | 73 |
| HCDR2 | dAb7r22 | TISPFGSTTYYADSVKG | 73 |
| HCDR2 | dAb7r23 | TISPFGSTTYYADSVKG | 73 |
| HCDR2 | dAb7r24 | TISPFGSTTYYADSVKG | 73 |
| HCDR2 | dAb7r25 | TISPFGSTTYYADSVKG | 73 |
| HCDR2 | dAb7r26 | TISPFGSTTYYADSVKG | 73 |
| HCDR2 | dAb7r27 | TISPFGSTTYYADSVKG | 73 |
| HCDR2 | dAb7r28 | TIHQTGFSTYYADSVKG | 74 |
| HCDR2 | dAb7r29 | MISSSGLWTYYADSVKG | 75 |
| HCDR2 | dAb7r30 | LIKPNGSPTYYADSVKG | 76 |
| HCDR2 | dAb7r31 | WIRPDGTFTYYADSVKG | 77 |
| HCDR2 | dAb7r32 | FIGREGYGTYYADSVKG | 78 |
| HCDR2 | dAb7r33 | SISSWGTGTYYADSVKG | 79 |
| HCDR2 | dAb7h30 | SITSSGSTYYADSVKG | 80 |
| HCDR2 | dAb7h31 | TITGTGAATYYADSVKG | 81 |

TABLE 11

| | | | SEQ ID |
|---|---|---|---|
| CDR | Clone # | Sequence | NO: |
| LCDR3 | dAb7m12 | QQVALYPKT | 82 |
| LCDR3 | dAb7m16 | QQGARWPQT | 83 |
| LCDR3 | dAb7m26 | QQVRKVPQT | 84 |
| LCDR3 | dAb7r1 | QQRYRMPYT | 85 |
| LCDR3 | dAb7r3 | QQRYMQPFT | 86 |
| LCDR3 | dAb7r4 | QQRYLQPYT | 87 |
| LCDR3 | dAb7r5 | QQRYITYNT | 88 |
| LCDR3 | dAb7r7 | QQRYSSPYT | 89 |
| LCDR3 | dAb7r8 | QQTFSKPST | 90 |
| LCDR3 | dAb7r13 | QQKYLPPYT | 91 |
| LCDR3 | dAb7r14 | QQRYRVPYT | 92 |
| LCDR3 | dAb7r15 | QQTSQWPHT | 93 |
| LCDR3 | dAb7r16 | QQRYLSPYT | 94 |
| LCDR3 | dAb7r17 | QQRWRAPYT | 95 |
| LCDR3 | dAb7r18 | QQYHQMPRT | 96 |
| LCDR3 | dAb7r19 | QQTHNPPKT | 97 |
| LCDR3 | dAb7h1 | QQTYTVPPT | 98 |
| LCDR3 | dAb7h2 | QQTYAVPPT | 99 |
| LCDR3 | dAb7h6 | QQTYNVPPT | 100 |
| LCDR3 | dAb7h7 | QQTFAVPPT | 101 |
| LCDR3 | dAb7h8 | QQTYRLPPT | 102 |
| HCDR3 | dAb7r20 | GGKDFDY | 103 |
| HCDR3 | dAb7r21 | GNLEPFDY | 104 |
| HCDR3 | dAb7r22 | KLSNGEDY | 105 |
| HCDR3 | dAb7r23 | VVKDNTEDY | 106 |
| HCDR3 | dAb7r24 | NTGGKQFDY | 107 |
| HCDR3 | dAb7r25 | KTGPSSFDY | 108 |
| HCDR3 | dAb7r26 | RTENRGVSFDY | 109 |
| HCDR3 | dAb7r27 | SDVLKTGLDGFDY | 110 |
| HCDR3 | dAb7r28 | VRSMRPYKFDY | 111 |
| HCDR3 | dAb7r29 | GFRLFPRTFDY | 112 |
| HCDR3 | dAb7r30 | GRGRFNVLQFDY | 113 |
| HCDR3 | dAb7r31 | SYMGDRFDY | 114 |
| HCDR3 | dAb7r32 | SVASFDY | 115 |
| HCDR3 | dAb7r33 | GGQGSFDY | 116 |
| HCDR3 | dAb7h30 | VNSLYKEDY | 117 |
| HCDR3 | dAb7h31 | QNSRYRFDY | 118 |

In some embodiments, the SDAB comprises a LCDR1 having a sequence as shown in Table 9 or a sequence having at least 90% identity thereto, or a sequence having 1, 2, 3 amino acid residue difference therefrom, a corresponding LCDR2 having a sequence as shown in Table 10 or a sequence having at least 90% identity thereto, or a sequence having 1, 2, 3 amino acid residue difference therefrom, and a corresponding LCDR3 having a sequence as shown in Table 11 or a sequence having at least 90% identity thereto, or a sequence having 1, 2, 3 amino acid residue difference therefrom.

In some embodiments, the SDAB comprises a HCDR1 having a sequence as shown in Table 9 or a sequence having at least 90% identity thereto, or a sequence having 1, 2, 3 amino acid residue difference therefrom, a corresponding HCDR2 having a sequence as shown in Table 10 or a sequence having at least 90% identity thereto, or a sequence having 1, 2, 3 amino acid residue difference therefrom, and a corresponding HCDR3 having a sequence as shown in Table 11 or a sequence having at least 90% identity thereto, or a sequence having 1, 2, 3 amino acid residue difference therefrom.

In some embodiments, the SDAB comprises a VH comprising the sequences listed in Table 12, or sequences having at least 80% (e.g. at least 85%, 88%, 90%, 91%, 92%, 93%, 94%, 95%, 96%, 97%, 98%, or 99%) identity thereto or sequences having 1, 2, 3, 4, 5, 6, 7, 8, 9, 10 amino acid residue difference therefrom. In some embodiments, the SDAB comprises a VL comprising the sequences listed in Table 12, or sequences having at least 80% (e.g. at least 85%, 88%, 90%, 91%, 92%, 93%, 94%, 95%, 96%, 97%, 98%, or 99%) identity thereto or sequences having 1, 2, 3, 4, 5, 6, 7, 8, 9, 10 amino acid residue difference therefrom. In some embodiments, the difference occur in regions outside the CDRs (e.g., in the FRs).

TABLE 12

| | | |
|---|---|---|
| VH1 | SEQ ID | EVQLLESGGGLVQPGGSLRLSCAASGFTFRTYTMAWVRQAPGKGL |
| | NO: 119 | EWVSSITSSGSTYYADSVKGRFTISRDNSKNTLYLQMNSLRAEDTA |
| | | VYYCAKVNSLYKEDYWGQGTLVTVSS |
| VH2 | SEQ ID | EVQLLESGGGLVQPGGSLRLSCAASGFTFRPTNMSWVRQAPGKGLE |
| | NO: 120 | WVSTITGTGAATYYADSVKGRFTISRDNSKNTLYLQMNSLRAEDTA |
| | | VYYCAKQNSRYRFDYWGQGTLVTVSS |
| VL1 | SEQ ID | DIQMTQSPSSLSASVGDRVTITCRASQSIFRHLKWYQQKPGKAPKLL |
| | NO: 121 | IYAASRLQSGVPSRFSGSGSGTDFTLTISSLQPEDFATYYCQQVALYP |
| | | KTFGQGTKVEIKR |
| VL2 | SEQ ID | DIQMTQSPSSLSASVGDRVTITCRASQSISSYLNWYQQKPGKAPKLLI |
| | NO: 122 | YRNSPLQSGVPSRFSGSGSGTDFTLTISSLQPEDFATYYCQQTYRVPP |
| | | TFGQGTKVEIKR |

In some embodiments, the single domain antibody is a nanobody where the single variable domain is derived from the variable domain of a camelid antibody (VHH domain), or the variable domain of cartilaginous fish antibody (VNAR domain). Both camelid antibodies and cartilaginous fish antibodies naturally lack light chains and consist of a pair of heavy chains.

In some embodiments, the nanobody comprises a CDR1 having a sequence as shown in Table 13, or a sequence having at least 90% identity thereto, or a sequence having 1, 2, 3 amino acid residue difference therefrom; a CDR2 having a sequence as shown in Table 14, or a sequence having at least 90% identity thereto, or a sequence having 1, 2, 3 amino acid residue difference therefrom; and a CDR3 having a sequence as shown in Table 15, or a sequence having at least 90% identity thereto, or a sequence having 1, 2, 3 amino acid residue difference therefrom.

TABLE 13

| CDR | Clone # | Sequence | SEQ ID NO: |
|---|---|---|---|
| HCDR1 | Clone 1 | TTCMA | 123 |
| HCDR1 | Clone 2 | DYTTG | 124 |

TABLE 14

| CDR | Clone # | Sequence | SEQ ID NO: |
|---|---|---|---|
| HCDR2 | Clone 1 | TITTGGTYPYYADSVLG | 125 |
| HCDR2 | Clone 2 | CISRSDGNTYYAESVL | 126 |

TABLE 15

| CDR | Clone # | Sequence | SEQ ID NO: |
|---|---|---|---|
| HCDR3 | Clone 1 | DASWGCRLSGSWSTVYNY | 127 |
| HCDR3 | Clone 2 | ADRYRSGFLGNGYEYD | 128 |

In some embodiments, the nanobody comprises a HCDR1 having a sequence as shown in Table 13 or a sequence having at least 90% identity thereto, or a sequence having 1, 2, 3 amino acid residue difference therefrom, a corresponding HCDR2 having a sequence as shown in Table 14 or a sequence having at least 90% identity thereto, or a sequence having 1, 2, 3 amino acid residue difference therefrom, and a corresponding HCDR3 having a sequence as shown in Table 15 or a sequence having at least 90% identity thereto, or a sequence having 1, 2, 3 amino acid residue difference therefrom.

In some embodiments, the nanobody comprises a VHH comprising the sequences listed in Table 16, or sequences having at least 80% (e.g. at least 85%, 88%, 90%, 91%, 92%, 93%, 94%, 95%, 96%, 97%, 98%, or 99%) identity thereto or sequences having 1, 2, 3, 6, 7, 8, 9, 10 amino acid residue difference therefrom. In some embodiments, the difference occur in regions outside the CDRs (e.g., in the FRs).

TABLE 16

| | |
|---|---|
| Clone #1 | VHH (SEQ ID NO: 129) |
| | QVQLQGSGGGSVQAGGSLRLSCAASAYTYSTTCMAWFRQAP |
| | GKEREKVATITTGGTYPYYADSVKGRFTISQDNAKNTVYLQ |
| | MNSLKPEDTAMYYCAADASWGCRLSGSWSTVYNYWGQGTQV |
| | TVSS |
| Clone #2 | VHH (SEQ ID NO: 130) |
| | DVQLQASGGDLVQAGGSLRLSCHASGLDDYIIGWFRQAPGK |
| | EREGLSCISRSDGNTYYAESVKGRFTISSDNAKNTVYLQMD |
| | SLKPEDTAVYYCAADRYRSGFLGNGYEYDYWGQGTQVTVSS |

In some embodiments, the extracellular domain further comprises a signal peptide. The term “signal peptide” as used herein refers to peptide, usually having a length of 5-30 amino acid residues, present at the N-terminus of a polypeptide that necessary for the translocation cross the membrane on the secretory pathway and control of the entry of the polypeptide to the secretory pathway.

In some embodiments, the signal peptide comprises a signal peptide of CD8 alpha: In some embodiments, the signal peptide of CD8 alpha comprises a sequence of SEQ ID NO: 130 or a sequence having at least 80%, 85%, 90%, 91%, 92%, 93%, 94%, 95%, 96%, 97%, 98%, 99% sequence identity thereto. In some embodiments, the signal peptide comprises a signal peptide of IgG.

It is also contemplated that the extracellular domain may be multi-specific or multivalent by multimerizing the antigen-binding domain that bind either the same antigen (multi-valent) or a different antigen (multi-specific).

In some embodiments, the CAR comprises a second antigen-binding domain. The second antigen-binding domain can be any domain that binds to the antigen including but not limited to an antibody or a fragment thereof (e.g., a Fv, a Fab, a (Fab)2, a scFv, a SDAB, a nanobody), and to an alternative scaffold known in the art to function as antigen binding domain.

In some embodiments, the second antigen-binding domain specifically binds to a different epitope of albumin from the first antigen-binding domain. CARs comprising antigen-binding domains that recognizes different epitopes are allowed to bind an albumin at the same time and promote ligand-induced dimerization of CARs.

In some embodiments, the second antigen binding domain binds specifically to a cancer antigen. Term “cancer antigen” refers to an antigenic substance produced in tumor. A cancer antigen expressed by both normal cells and cancer cells, overexpressed in a cancer cell in comparison to a normal cell, or expressed exclusively on the cell surface of a cancer cell. In some embodiments, the cancer antigen is selected from CD19, CD20, CAIX, CD33, CD44v7/8, CEA, EGP-2, EGP-40, erb-B2, erb-B3, erb-B4, FBP, fetal acetyl choline receptor, GD2, GD3, Her2/neu, IL-13R-a2, KDR, k-light chain, LeY, LI cell adhesion molecule, MAGE-A1, mesothelin, MUCI, KG2D ligands, oncofetal antigen (h5T4), PSCA, PSMA, TAA targeted by mAb IgE, TAG-72, and VEGF-R.

In some embodiments, the first and the second antigen-binding domain are arranged in tandem, optionally separated by a polypeptide linker (FIG. 1B). The polypeptide linker can be of at least 1, 2, 3, 4, 5, 6, 7, 8, 9, 10, 11, 12, 13, 14, 15, 16, 17, 18, 19, 20, 25, 30, 35, 40, 45, 50, or more amino acid residues. The linker sequence may comprise any naturally occurring amino acid. In some embodiments, the linker sequence comprises amino acids glycine and serine.

### Transmembrane Domain

The transmembrane domain of the CAR described herein may be derived from any membrane-bound or transmembrane protein including, but are not limited to, BAFFR, BLAME (SLAMF8), CD2, CD3 epsilon, CD4, CD5, CD8, CD9, CD11a (CD18, ITGAL, LFA-1), CD11b, CD11c, CD11d, CD16, CD19, CD22, CD27, CD28, CD29, CD33, CD37, CD40, CD45, CD49a, CD49d, CD49f, CD64, CD80, CD84, CD86, CD96 (Tactile), CD100 (SEMA4D), CD103, CD134, CD137 (4-1BB), CD150 (IPO-3, SLAMF1, SLAM), CD154, CD160 (BY55), CD162 (SELPLG), CD226 (DNAM1), CD229 (Ly9), CD244 (2B4, SLAMF4), CD278 (ICOS), CEACAM1, CRT AM, GITR, HYEM (LIGHTR), IA4, IL2R beta, IL2R gamma, IL7R a, ITGA1, ITGA4, ITGA6, ITGAD, ITGAE, ITGAM, ITGAX, ITGB1, ITGB2, ITGB7, KIR, LTBR, OX40, NKG2C, NKG2D, NKp30, NKp44, NKp46, NKp80 (KLRF1), PAG/Cbp, PSGL1, SLAMF6 (NTB-A, Ly108), SLAMF7, an alpha, beta or zeta chain of a T-cell receptor, TNFR2, VLA1, and VLA-6.

In one embodiment, the CAR described herein comprises a transmembrane domain of CD8 alpha, CD28 or ICOS. In certain embodiments, the transmembrane domain of CD8 alpha has a sequence of SEQ ID NO: 132, or a sequence having at least 80%, 85%, 90%, 91%, 92%, 93%, 94%, 95%, 96%, 97%, 98%, 99% sequence identity thereto.

In certain embodiments, the transmembrane domain of the CAR described herein is synthetic, e.g., comprising predominantly hydrophobic residues such as leucine and valine. In certain embodiment, the transmembrane domain of the CAR described herein is modified or designed to avoid binding to the transmembrane domains of the same or different surface membrane proteins in order to minimize interactions with other members of the receptor complex.

In some embodiments, the CAR described herein further comprises a hinge region, which forms the linkage between the extracellular domain and transmembrane domain of the CAR. The hinge and/or transmembrane domain provides cell surface presentation of the extracellular domain of the CAR.

The hinge region may be derived from any membrane-bound or transmembrane protein including, but are not limited to, BAFFR, BLAME (SLAMF8), CD2, CD3 epsilon, CD4, CD5, CD8, CD9, CD11a (CD18, ITGAL, LFA-1), CD11b, CD11c, CD11d, CD16, CD19, CD22, CD27, CD28, CD29, CD33, CD37, CD40, CD45, CD49a, CD49d, CD49f, CD64, CD80, CD84, CD86, CD96 (Tactile), CD100 (SEMA4D), CD103, CD134, CD137 (4-1BB), CD150 (IPO-3, SLAMF1, SLAM), CD154, CD160 (BY55), CD162 (SELPLG), CD226 (DNAM1), CD229 (Ly9), CD244 (2B4, SLAMF4), CD278 (ICOS), CEACAM1, CRT AM, GITR, HYEM (LIGHTR), IA4, IL2R beta, IL2R gamma, IL7Ra, ITGA1, ITGA4, ITGA6, ITGAD, ITGAE, ITGAM, ITGAX, ITGB1, ITGB2, ITGB7, KIR, LTBR, OX40, NKG2C, NKG2D, NKp30, NKp44, NKp46, NKp80 (KLRF1), PAG/Cbp, PSGL1, SLAMF6 (NTB-A, Ly108), SLAMF7, an alpha, beta or zeta chain of a T-cell receptor, TNFR2, VLA1, and VLA-6.

In some embodiments, the hinge region comprises a hinge region of CD8 alpha, a hinge region of human immunoglobulin (Ig), or a glycine-serine rich sequence.

In some embodiments, the CAR comprises a hinge region of CD8 alpha, CD28, ICOS or IgG4m. In certain embodiments, the hinge region has a sequence of SEQ ID NO: 133, or a sequence having at least 80%, 85%, 90%, 91%, 92%, 93%, 94%, 95%, 96%, 97%, 98%, 99% sequence identity thereto.

### Intracellular Domain

The intracellular domain of the CAR described herein, is responsible for activation of at least one of the normal effector functions of the immune cell in which the CAR has been placed in. The term “effector function” used in the context of an immune cell refers to a specialized function of the cell, for example, the cytolytic activity or helper activity including the secretion of cytokines for a T cell.

It is well recognized that the full activation of a T-cell requires signals generated through the T-cell receptor (TCR) and a secondary or co-stimulatory signal. Thus, the T cell activation is mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

The intracellular domain of the CAR can be derived from a molecule which transduces the effector function signal and directs the cell to perform the effector function, or a truncated portion of such molecule as long as it transduces the signal. Such protein molecule including, but are not limited to, B7-H3, BAFFR, BLAME (SLAMF8), CD2, CD3 delta, CD3 epsilon, CD3 gamma, CD3 zeta, CD4, CD5, CD7, CD8alpha, CD8beta, CD11a (CD18, LFA-1, ITGAL), CD11b, CD11c, CD11d, CD19, CD27, CD28, CD29, CD30, CD40, CD49a, CD49d, CD49f, CD69, CD79a, CD79b, CD83, CD84, CD86, CD96 (Tactile), CD100 (SEMA4D), CD103, CD127, CD137 (4-1BB), CD150 (SLAM, SLAMF1, IPO-3), CD160 (BY55), CD162 (SELPLG), CD226 (DNAM1), CD229 (Ly9), CD244 (SLAMF4, 2B4), CEACAM1, CRTAM, DAP10, DAP12, common FcR gamma, FcR beta (Fc Epsilon Rib), Fcgamma RIIa, GADS, GITR, HVEM (LIGHTR), IA4, IL2R beta, IL2R gamma, IL7R alpha, ITGA4, ITGA6, ITGAD, ITGAE, ITGAM, ITGAX, ITGB1, ITGB2, ITGB7, ICAM-1, ICOS, LIGHT, LTBR, LAT, NKG2C, NKG2D, NKp44, NKp30, NKp46, NKp80 (KLRF1), OX40, PD-1, PAG/Cbp, PSGL1, SLP-76, SLAMF6 (NTB-A, Ly108), SLAMF7, T cell receptor (TCR), TNFR2, TRANCE/RANKL, VLA1, VLA-6, any derivative, variant, or fragment thereof, any synthetic sequence of a molecule that has the same functional capability, and any combination thereof.

In some embodiments, the intracellular domain comprises a co-stimulatory domain and a signaling domain, wherein upon binding of the CAR to albumin, the co-stimulatory domain provides co-stimulatory intracellular signaling without the need of its original ligand, and the signaling domain provides the primary activation signaling. The co-stimulatory domain and the signaling domain of the CAR can be linked to each other in a random or specified order.

### Co-Stimulator Domain

In some embodiments, the co-stimulatory domain is derived from an intracellular domain of a co-stimulatory molecule.

Examples of co-stimulatory molecules include B7-H3, BAFFR, BLAME (SLAMF8), CD2, CD4, CD8 alpha, CD8 beta, CD7, CD11a, CD11b, CD11c, CD11d, CD 18, CD19, CD27, CD28, CD29, CD30, CD40, CD49a, CD49D, CD49f, CD69, CD83, CD84, CD96 (Tactile), CD100 (SEMA4D), CD103, CD127, CD137(4-1BB), CD150 (SLAM, SLAMF1, IPO-3), CD160 (BY55), CD162 (SELPLG), CD226 (DNAM1), CD229 (Ly9), CD244 (SLAMF4, 2B4), CEACAM1, CRTAM, CDS, OX40, PD-1, ICOS, GADS, GITR, HVEM (LIGHTR), IA4, ICAM-1, IL2R beta, IL2R gamma, IL7R alpha, ITGA4, ITGA4, ITGA6, ITGAD, ITGAE, ITGAL, ITGAM, ITGAX, ITGB1, ITGB2, ITGB7, LAT, LFA-1, LIGHT, LTBR, NKG2C, NKG2D, NKp44, NKp30, NKp46, NKp80 (KLRF1), PAG/Cbp, PSGL1, SLAMF6 (NTB-A, Lyl08), SLAMF7, SLP-76, TNFR2, TRANCE/RANKL, VLA1, VLA-6, any derivative, variant, or fragment thereof, any synthetic sequence of a co-stimulatory molecule that has the same functional capability, and any combination thereof.

In some embodiment, the co-stimulatory domain of the CAR comprises an intracellular domain of co-stimulatory molecule CD137 (4-1BB), CD28, OX40 or ICOS. In some embodiments, the co-stimulatory domain of the CAR has a sequence of SEQ ID NO: 134. or a sequence having at least 80%, 85%, 90%, 91%, 92%, 93%, 94%, 95%, 96%, 97%, 98%, 99% sequence identity thereto.

### Signaling Domain

The primary activation of the TCR complex can be regulated by a primary cytoplasmic signaling sequence either in a stimulatory manner or in an inhibitory manner. Primary cytoplasmic signaling sequences that act in a stimulatory manner may contain signaling motifs known as immunoreceptor tyrosine-based activation motifs (ITAMs). Examples of ITAM containing primary signaling sequences that are of particular use in the disclosure include those derived from CD3 gamma, CD3 delta, CD3 epsilon, CD3 zata, CD5, CD22, CD79a, CD79b, CD66d, FcR gamma, FcR beta, and TCR zeta.

In some embodiments, the signaling domain of the CAR of the disclosure comprises an ITAM that provides stimulatory intracellular signaling upon the CAR binding to albumin, without HLA restriction. In some embodiments, the signaling domain of the CAR comprises a signaling domain of CD3 zeta (CD247). In some embodiments, the signaling domain of the CAR comprises a sequence of SEQ ID NO: 135, or a sequence having at least 80%, 85%, 90%, 91%, 92%, 93%, 94%, 95%, 96%, 97%, 98%, 99% sequence identity thereto.

### Other Region

In some embodiments, the CAR further comprises a linker. The term “linker” as provided herein is a polypeptide connecting various components of the CAR.

In some embodiment, the linker is inserted between the VH and VL of the scFv. In some embodiments, the linker is inserted between the transmembrane domain and the intracellular domain. In some embodiments, the linker is between the signaling domain and the co-stimulatory domain of the intracellular domain.

In some embodiments, the linker comprises a glycine-serine (GS) doublet between 2 and 20 amino acid residues in length. Exemplary GS doublets include (G₄S)₃: SEQ ID NO: 136. In some embodiments, the polynucleotide provided herein comprises a nucleotide sequence encoding a linker.

In some embodiments, the CAR provided herein comprises from the N-terminus to the C-terminus: a signal peptide of CD8 alpha, an antigen binding domain specifically binds to albumin, a hinge region of CD8 alpha, a transmembrane domain of CD8 alpha, an intracellular domain of CD137, and a signaling domain of CD3 zeta.

In some embodiments, the polynucleotide provided herein encodes a CAR comprising from the N-terminus to the C-terminus: a signal peptide of CD8 alpha, an antigen binding domain specifically binds to albumin, a hinge region of CD8 alpha, a transmembrane domain of CD8 alpha, an intracellular domain of CD137, and a signaling domain of CD3 zeta.

In some embodiments, the CAR demonstrates a high affinity to albumin. The term “affinity” as used herein refers to the strength of non-covalent interaction between an immunoglobulin molecule or fragment thereof and an antigen. The binding affinity can be represented by Kd value, i.e., the dissociation constant, determined by any methods known in the art, including, without limitation, enzyme-linked immunosorbent assays (ELISA), surface plasmon resonance, or flow cytometry (such as FACS). In certain embodiments, the CAR has a binding affinity to albumin of less than 50 nM, 25 nM, 10 nM, 5 nM, 4 nM, 3 nM, 2 nM, or 1 nM.

In some embodiments, the CAR has the structure: S-AB-H-TM-IC wherein S is a signal peptide, AB is the antigen-binding domain, H is a hinge region, TM is the transmembrane domain and IC is the intracellular signaling domain. In some embodiments, the CAR has the structure: S-VH-L-VL-H-TM-IC, wherein VH is a variable heavy region, L is a linker, VL is a variable light region. In some embodiments, the CAR has the structure: S-VL-L-VH-H-TM-IC, wherein VH is a variable heavy region, L is a linker, VL is a variable light region.

In some embodiments, the CAR has the structure: S-SDAB-TM-IC, wherein SDAB is a single domain antibody.

In some embodiments, the CAR has the structure: S-N-TM-IC, wherein N is a nanobody.

In some embodiments, the CAR has the structure: S-AB1-L-AB2-H-TM-IC or S-AB2-L-AB1-H-TM-IC, wherein AB1 is the first antigen-binding domain specifically binds to albumin, and AB2 is the second antigen-binding domain specifically binds to a different epitope of albumin from the first antigen-binding domain or to a cancer antigen. In some embodiments, AB1 and AB2 can independently be a scFV, a SDAB or a nanobody.

### Vector

In another aspect, the present disclosure provides a vector comprising the polynucleotide encoding the CAR as described herein. The polynucleotides encoding a CAR can be inserted into different types of vectors known in the art, for example, a plasmid, a phagemid, a phage derivative, a viral vector derived from animal virus, a cosmid, transposon, a site directed insertion vector (e.g., CRISPR, Zinc finger nucleases, TALEN), an in vitro transcribed RNA, or a suicide expression vector. In some embodiments, the vector is a DNA or RNA.

In some embodiment, the polynucleotide is operatively linked to at least one regulatory polynucleotide element in the vector for expression of the CAR. Typical vectors contain various regulatory polynucleotide elements, for example, elements (e.g., transcription and translation terminators, initiation sequences, and promoters) regulating the expression of the inserted polynucleotides, elements (e.g., origin of replication) regulating the replication of the vector in a host cell, and elements (e.g., terminal repeat sequence of a transposon) regulating the integration of the vector into a host genome. The expression of the CAR can be achieved by operably linking the polynucleotides encoding a CAR to a promoter, and incorporating the construct into a vector. Both constitutive promoters (such as a CMV promoter, a SV40 promoter, and a MMTV promoter), or inducible promoters (such as a metallothionine promoter, a glucocorticoid promoter, and a progesterone promoter) are contemplated for the disclosure. In some embodiment, the vector is an expression vector, an expression vector comprises sufficient cis-acting elements for expression; other elements for expression can be supplied by the host cell or in an in vitro expression system.

In order to assess the expression of a CAR, the vector can also comprise a selectable marker gene or a reporter gene or both for identification and selection of the cells to which the vector are introduced. Useful selectable markers include, for example, antibiotic-resistance genes, such as neo and the like. Useful reporters include, for example, luciferase, beta-galactosidase, chloramphenicol acetyl transferase, secreted alkaline phosphatase, or the green fluorescent protein gene.

In some embodiments, the vector is a viral vector. Viral vectors may be derived from, for example, retroviruses, adenoviruses, adeno-associated viruses (AAV), herpes viruses, and lentiviruses. Useful viral vectors generally contain an origin of replication functional in at least one organism, a promoter, restriction endonuclease sites, and one or more selectable markers. In some embodiments, the vector is a retrovirus vector, such as lentiviral vector. Lentiviral vector is particular useful for long-term, stable integration of the polynucleotide encoding the CAR into the genome of non-proliferating cells that result in stable expression of the CAR in the host cell, e.g., host T cell.

In some embodiments, the vector is RNA (e.g., mRNA). As the RNA would dilute out with cell division, the expression of the RNA would not be permanent. In one embodiment, the in vitro transcribed RNA CAR can be introduced to a cell as a form of transient expression (FIG. 1D).

Chemical structures with the ability to promote stability and/or translation efficiency may also be used in an RNA. A method for generating RNA for use in transfection can involve in vitro transcription (IVT) of a template with specially designed primers, followed by polyA addition, to produce a construct containing 3′ and 5 untranslated sequence (“UTR”), a 5′ cap and/or Internal Ribosome Entry Site (IRES), the nucleic acid to be expressed, and a polyA tail typically 50-2000 bases in length. RNA so produced can efficiently transfect different kinds of cells.

RNA can be introduced into target cells using any of a number of different methods, for instance, available methods which include, but are not limited to, electroporation or the Gene Pulser II (BioRad, Denver, Colo.), Multiporator (Eppendort, Hamburg Germany), cationic liposome mediated transfection using lipofection, polymer encapsulation, peptide mediated transfection, orbiolistic particle delivery systems such as “gene guns”.

In some embodiments, the vector is expression DNA vector (e.g., plasmid, virus). When the expression DNA vector introduced into the cell transiently, mRNA of the CAR will be transcribed in host cell. As the DNA vector and the mRNA would dilute out with cell division, the expression of the RNA would not be permanent. In one embodiment, the DNA vector can be introduced to a cell as a form of transient expression of the CAR.

In some embodiments, the vector is a transposon-based expression vector. A transposon is a DNA sequence that can change its position within a genome. In a transposon system, the polynucleotide encoding the CAR is flanked by terminal repeat sequences recognizable by a transposase which mediates the movement of the transposon. A transposase can be co-delivered as a protein, encoded on the same vector as the CAR, or encoded on a separate vector. Non-limiting examples of transposon systems include Sleeping Beauty, Piggyback, Frog Prince, and Prince Charming.

A vector can be introduced into a host cell, e.g., mammalian cell by any method known in the art, for example, by physical, chemical or biological means. Physical methods for introducing a polynucleotide into a host cell include calcium phosphate precipitation, lipofection, particle bombardment, microinjection, electroporation, and the like. Biological methods include the use of viral vectors, and especially retroviral vectors, for inserting genes into mammalian, e.g., human cells. Chemical means include colloidal dispersion systems, such as macromolecule complexes, nanocapsules, microspheres, beads, and lipid-based systems including oil-in-water emulsions, micelles, mixed micelles, and liposomes.

### Cells

In one aspect, the disclosure provides an engineered cell comprising or expressing the CAR as described here. In some embodiments, the engineered cell comprises the polynucleotide encoding the CAR, or the vector comprising the CAR polynucleotide.

In some embodiments, the engineered cell further comprises or expresses a second CAR (FIG. 1B). In some embodiments, the second CAR comprises a second antigen-binding domain specifically binding to a different epitope of albumin from the first antigen-binding domain. In some embodiments, the second CAR comprises a second antigen-binding domain specifically binding to a cancer antigen. In some embodiments, the cancer antigen is selected from the group consisting of CD19, CD20, CAIX, CD33, CD44v7/8, CEA, EGP-2, EGP-40, erb-B2, erb-B3, erb-B4, FBP, fetal acetyl choline receptor, GD2, GD3, Her2/neu, IL-13R-a2, KDR, k-light chain, LeY, LI cell adhesion molecule, MAGE-A1, mesothelin, MUCI, KG2D ligands, oncofetal antigen (h5T4), PSCA, PSMA, TAA, TAG-72, and VEGF-R.

In some embodiments, a dual CAR-T cell may be generated by introducing and expressing a vector comprising nucleotides encoding both the first CAR and the second CAR. In some embodiments, a dual CAR-T cell may be generated by introducing and expressing a first vector comprising nucleotides encoding the first CAR and a second vector comprising nucleotides encoding the second CAR.

In some embodiments, the first vector and the second vector are of the same type e.g., a plasmid, a phagemid, a phage derivative, a viral vector derived from animal virus, a cosmid, transposon, a site directed insertion vector (e.g., CRISPR, Zinc finger nucleases, TALEN), an in vitro transcribed RNA, or a suicide expression vector. In some embodiments, the first vector and the second vector are of different types. In some embodiments, the first vector and second vector are independently selected from vectors that enable either permanent or transient expression. In some embodiments, the CAR comprising the first-antigen binding domain specific for albumin is expressed from a RNA vector (e.g., an in vitro transcribed RNA), and the CAR comprising the second-antigen binding domain specific for a cancer antigen is expressed from a non-RNA vector.

An engineered cell as described herein is a genetically modified immune cell, Immune cells useful for the disclosure include T cells, natural killer (NK) cells, invariant NK cells, or NKT cells, and other effector cell. In some embodiment, the immune cells are primary cells, expanded cells derived from primary cells, or cells derived from stem cells differentiated in vitro. “T cell” includes all types of immune cells expressing CD3, including, e.g., T-helper cells (CD4+ cells), cytotoxic T-cells (CD8+ cells), T-regulatory cells (Treg) and gamma-delta T cells.

In another aspect, the disclosure provides a method of making an engineered cell expressing the CAR as described herein. In some embodiments, the method comprising one of more steps selected from of obtaining cells from a source, culturing cells, activating cells, expanding cells and engineering cells

In another aspect, the disclosure provides a method of using the engineered cells for cell therapy, wherein the engineered cells are introducing into a subject. In some embodiments, the subject is the same subject from who the cells are obtained.

### Sources of Cells

The engineered cells can be derived from immune cells isolated from subjects, e.g., human subjects. In some embodiments, the immune cells are obtained from a subject of interest, such as a subject suspected of having a particular disease or condition, a subject suspected of having a predisposition to a particular disease or condition, a subject who will undergo, is undergoing, or have undergone treatment for a particular disease or condition, a subject who is a healthy volunteer or healthy donor, or from blood bank. Thus, the cells can be autologous or allogeneic to the subject of interest. Allogeneic donor cells may not be human-leukocyte-antigen (HLA)-compatible, and thus allogeneic cells can be treated to reduce immunogenicity.

Immune cells can be collected from any location in which they reside in the subject including, but not limited to, blood, cord blood, spleen, thymus, lymph nodes, pleural effusion, spleen tissue, tumor and bone marrow. The isolated immune cells may be used directly, or they can be stored for a period of time, such as by freezing.

In some embodiments, the engineered cells are derived from T cells. T cells can be obtained from blood collected from a subject using any number of techniques known to the skilled artisan, such as apheresis.

In some embodiments, one or more of the T cell populations is enriched for or depleted of cells that are positive for a specific marker, such as surface markers, or that are negative for a specific marker. Such markers are those that are absent or expressed at relatively low levels on certain populations of T cells but are present or expressed at relatively higher levels on certain other populations of T cells. In some embodiments, CD4+ helper and CD8+ cytotoxic T cells are isolated. In some embodiments, CD8+ and CD4+ T cells are further enriched for or depleted of naive, central memory, effector memory, and/or central memory stem cells, such as by positive or negative selection based on surface antigens associated with the respective subpopulation.

### Activation and Expansion of Cells

Immune cell activation and/or expansion is one of the major step for immune cell function. In some embodiments, the immune cells are activated and expanded at the same, prior to or/and after genetic modification. In some embodiment, the immune cells are activated and/or expanded in vitro, ex vivo or in vivo.

Method for activation and expansion of immune cells have been described in the art and can be used in the methods described herein. For example, the T cells can be activated and expanded by contacting with a surface having attached thereto an agent that stimulates a CD3/TCR complex associated signal and a ligand that stimulates a co-stimulatory molecule on the surface of the T cells. In particular, T cell populations may be stimulated, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. In certain embodiments, the primary stimulatory signal and the co-stimulatory signal for the T cell may be provided by different protocols.

In some aspects, the method further comprises expanding and/or inducing proliferation of T cells in vitro or ex vivo by contacting the engineered cell of the disclosure with a composition comprising albumin. In some embodiments, the engineered cell is a T cell. Contacting the engineered cell of the disclosure with a composition comprising albumin can improve the current immune cell culture process and increase the percentage of CAR-positive T cells during the culture.

In some embodiments, the composition comprises 0.1-10 mg/mL of albumin. In some embodiments, the composition comprises at least, at most, or about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 10 mg/mL of albumin (or any range derivable therein).

In some embodiments, the composition further comprises IL-2. In some embodiments, the composition comprises 20-400 U/mL of IL-2. In some embodiments, the composition comprises at least, at most, or about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600 U/mL of JL-2 (or any range derivable therein).

In some embodiments, the method further comprises contacting the cells with feeder cells. In some embodiments, the feeder cells are irradiated. Feeder cells or support cells can include, for example, fibroblasts, mouse embryonic fibroblasts, JK1 cells, SNL 76/7 cells, human fetal skin cells, human fibroblasts, human PBMC, and human foreskin fibroblasts.

In some embodiments, the method excludes contacting T cells with feeder cells. In some cases, the excluded feeder cells are from a different animal species as the T cells.

In some aspects, the method comprises expanding and/or inducing proliferation of T cells in vivo by contacting the engineered cell of the disclosure with albumin in blood. In some embodiments, the engineered cell is a T cell.

### Method of Stimulating an Immune Response

In another aspect, the current disclosure relate to methods for stimulating an immune response. The immune response stimulation may be carried out in vitro, ex vivo or in vivo. In some embodiments, the methods relate to cells as described herein capable of stimulating an immune response in the presence of albumin.

In some embodiments, stimulating an immune response comprises increasing expression and/or secretion of immune stimulating cytokines and/or molecules. In some embodiments, the immune stimulating cytokines and/or molecules are one or more of TNF-a, IFN-β, IFN-γ, IL-1, IL-2, IL-4, IL-6, IL-8, IL-10, IL-12, IL-18 and granulocyte-macrophage colony stimulating factor.

In some embodiments, stimulating an immune response comprises increasing proliferation of immune cells. In some embodiments, the immune cells are T cells.

An increase in expression or proliferation as described herein may be at least, at most, or exactly 1, 2, 3, 4, 5, 6, 7, 8, 9, 10, 50, 100, 200, 300, 500, or 1000 fold increase over a base-line expression level such as a control (non-disease, non-albumin or non-antigen binding polypeptide control).

In some embodiments, the stimulation is carried out in vitro or ex vivo, where the engineered cell as herein described is contacted with a composition comprising albumin.

In some embodiments, the stimulation is carried out in vivo, wherein the engineered cell as herein described is contacted with endogenous albumin produced in a human subject in need of immune stimulation. In some embodiments, the method comprises administering a cell described herein comprising the CAR or nucleic acids of the disclosure to a human subject (e.g., the individual from whom the cell was obtained) and the genetically modified cell is activated in vivo (i.e., by endogenously produced albumin).

Stimulation of immune response in vivo can increase the proliferation of immune cells in vivo, making the in vivo cell expansion more effective, shortening the time for in vitro cell culture and reducing cost of goods. In some embodiment, the in vivo expansion of the engineered the cells as described herein can be at least 2, 5, 10, 20, 30, 40, 50, 100 times more effective than a control (e.g., non-engineered immune cells or engineered cells expressing non-albumin binding CAR).

In some embodiments, the human subject has cancer. In some embodiments, the cancer is a solid cancer selected from the group consisting of adrenal cancer, bone cancer, brain cancer, breast cancer, colorectal cancer, esophageal cancer, eye cancer, gastric cancer, head and neck cancer, kidney cancer, liver cancer, lung cancer, non-small cell lung cancer, bronchioloalveolar cell lung cancer, mesothelioma, head and neck cancer, squamous cell carcinoma, melanoma, oral cancer, ovarian cancer, cervical cancer, penile cancer, prostate cancer, pancreatic cancer, skin cancer, sarcoma, testicular cancer, thyroid cancer, uterine cancer, vaginal cancer. In some embodiments, the cancer is a hematologic malignancy selected from the group consisting of acute lymphocytic leukemia (ALL), acute myeloid leukemia (AML), B-cell leukemia, chronic lymphoblastic leukemia (CLL), blastic plasmacytoid dendritic cell neoplasm (BPDCN), chronic myelomonocytic leukemia (CMML), chronic myelocytic leukemia (CML), pre-B acute lymphocytic leukemia (Pre-B ALL), diffuse large B-cell lymphoma (DLBCL), extranodal NK/T-cell lymphoma, hairy cell leukemia, heavy chain disease, HHV8-associated primary effusion lymphoma, plasmablastic lymphoma, primary CNS lymphoma, primary mediastinal large B-cell lymphoma, T-cell/histiocyte-rich B-cell lymphoma, Hodgkin's lymphoma, non-Hodgkin's lymphoma, Waldenstrom's macroglobulinemia, multiple myeloma (MM), myelodysplastic syndromes (MDS), myeloproliferative neoplasms, and polycythemia vera.

### Method of Treatment

In another aspect, the present disclosure provides a method for treating a disease or pathological condition in a patient comprising administering a therapeutically effective amount of the engineered cell provided herein to the patient. In some embodiments, the disease is cancer. In some embodiments, the cancer is a solid tumor or a hematologic malignancy as described herein.

In some embodiments, the method for treating a disease or pathological condition comprises providing T cells isolated from a subject, engineering the T cells to express the CAR as provided herein, and transfuse the engineered T cells back into the subject. In some embodiments, the method further comprises activating and/or expanding the engineered cells in vitro or ex vivo as described herein, e.g., by a method comprising contacting the engineered cell with a composition comprising albumin. In some embodiments, the composition further comprises IL-2. In some embodiments, the activating and/or expanding the engineered cells in vitro or ex vivo further comprise contacting the engineered cells with feeder cells. In some embodiments, the feeder cells are irradiated.

In certain embodiments, the treatment method further comprises administering an agent that increases the efficacy of the engineered cells. For example, a growth factor that promotes the growth and activation of the engineered cells of the present disclosure is administered to the subject either concomitantly with the cells or subsequently to the cells. The growth factor can be any suitable growth factor that promotes the growth and activation of the immune cells. Examples of suitable immune cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

In some embodiments, the treatment method further comprises administering an agent that reduces of ameliorates a side effect associated with the administration of the engineered cells. Exemplary side effects include cytokine release syndrome (CRS), and hemophagocytic lymphohistiocytosis (HLH, also termed macrophage activation syndrome (MAS)). The agent administered to treat the side effects can be an agent neutralizing soluble factors such as IFN-gamma, IFN-alpha, IL-2 and IL-6. Such agents include, without limitation, an inhibitor of TNF-alpha (e.g., entanercept) and an inhibitor of IL-6 (e.g., tocilizumab).

Therapeutically effective amounts of the engineered cells can be administered by a number of routes, including parenteral administration, for example, intravenous, intraperitoneal, intramuscular, intrasternal, or intraarticular injection, or infusion.

The engineered cells can be administered in various treatment regimens, for example, in a single or a few doses over one to several days or periodic doses over an extended time. The precise dose to be employed will also depend on the route of administration, and the seriousness of disease or pathological condition in the patient, and should be decided according to the judgment of the practitioner and each patient's circumstances. The therapeutically effective amount of engineered cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ cells/m2. In a certain embodiment, the dose used in the treatment of human subjects ranges from about 3.8×10⁹ to about 3.8×10¹⁰ cells/m². In additional embodiments, a therapeutically effective amount of the engineered cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of engineered cells is readily determined by one of skill in the art based on the age, weight, sex, and physiological condition of the subject. Effective doses can be extrapolated from dose-response curves derived from in vitro or animal model test systems.

In another aspect, the present disclosure also provides a pharmaceutical composition comprising the engineered cells and a pharmaceutically acceptable diluent and/or carrier. Exemplary diluent and/or carrier include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

TABLE 17
Exemplary sequences of domains comprised in CAR

| SEQ ID NO. | Sequence | Domain |
|---|---|---|
| 131 | MALPVTALLLPLALLLHAARP | CD8 alpha signal |
| | | peptide |
| 132 | DIQMTQSPSSLSASVGDRVTITCRASQSIFRHLK | albumin binding |
| | WYQQKPGKAPKLLIYAASRLQSGVPSRFSGSGS | domain |
| | GTDFTLTISSLQPEDFATYYCQQVALYPKTFGQ | |
| | GTKVEIKR | |
| 133 | IYIWAPLAGTCGVLLLSLVIT | CD8 alpha |
| | | transmembrane domain |
| 134 | TTTPAPRPPTPAPTIASQPLSLRPEACRPAA | CD8 alpha hinge |
| | GGAVHTRGLDFACD | region |
| 135 | KRGRKKLLYIFKQPFMRPVQTTQEEDGCSC | CD137 intracellular |
| | RFPEEEEGGCEL | domain |
| 136 | RVKFSRSADAPAYQQGQNQLYNELNLGRR | CD3 zeta (CD247) |
| | EEYDVLDKRRGRDPEMGGKPRRKNPQEGL | signaling domain |
| | YNELQKDKMAEAYSEIGMKGERRRGKGH | |
| | DGLYQGLSTATKDTYDALHMQALPPR | |
| 137 | GGGGSGGGGSGGGGS | (G₄S)₃ |

### EXAMPLE

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### Example 1

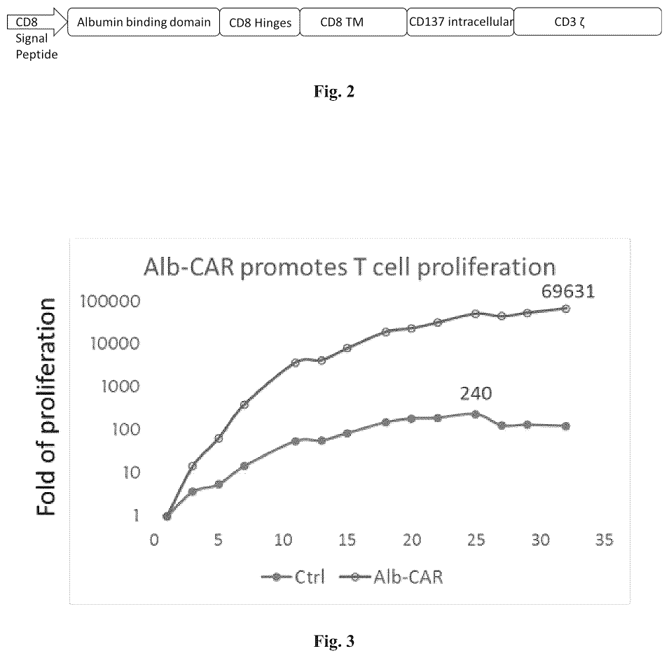

Example 1 illustrates that expressing of the CAR against albumin enhances T cell expansion ex vivo. Alb CAR construct was designed and generated as described in FIG. 2. The Alb CAR construct includes a CD8 signal peptide having an amino acid sequence of SEQ ID NO: 131 (MALPVTALLLPLALLLHAARP), an albumin binding domain having an amino acid sequence of SEQ ID NO: 139 (EVQLLESGGGLVQPGGSLRLSCAASGFTFWPYTMSWVRQAPGKGLEWVSTISPFGS TTYYADSVKGRFTISRDNSKNTLYLQMNSLRAEDTAVYYCAKSDVLKTGLDGFDY WGQGTLVTVSS), a CD8 hinge having an amino acid sequence of SEQ ID NO: 134 (TTTPAPRPPTPAPTIASQPLSLRPEACRPAAGGAVHTRGLDFACD), a CD8 transmembrane domain having an amino acid sequence of SEQ ID NO: 133 (IYIWAPLAGTCGVLLLSLVIT), a CD137 intracellular domain having an amino acid sequence of SEQ ID NO: 135 (KRGRKKLLYIFKQPFMRPVQTTQEEDGCSCRFPEEEEGGCEL), and an intracellular domain of CD3zeta having an amino acid sequence of SEQ ID NO: 136 (RVKFSRSADAPAYQQGQNQLYNELNLGRREEYDVLDKRRGRDPEMGGKPRRKNP QEGLYNELQKDKMAEAYSEIGMKGERRRGKGHDGLYQGLSTATKDTYDALHMQA LPPR).

The full-length amino acid sequence of the Alb CAR construct tested in the Example is shown below:

(SEQ ID NO: 138)
MALPVTALLLPLALLLHAARPEVQLLESGGGLVQPGGSLRLSCAASGFTF
WPYTMSWVRQAPGKGLEWVSTISPFGSTTYYADSVKGRFTISRDNSKNTL
YLQMNSLRAEDTAVYYCAKSDVLKTGLDGFDYWGQGTLVTVSSTTTPAPR
PPTPAPTIASQPLSLRPEACRPAAGGAVHTRGLDFACDIYIWAPLAGTCG
VLLLSLVITKRGRKKLLYIFKQPFMRPVQTTQEEDGCSCRFPEEEEGGCE
LRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKD
TYDALHMQALPPR

Human T-cell from healthy donor whole blood obtained from the TPCS. T-cells were stimulated with CD3/CD28 Dynabeads (Thermo Fisher Scientific) at a 1:1 cell:bead ratio for 24H then transduced with the lentivirus containing the CAR construct. T cells were cultured in XF T Cell Expansion Medium (STEMCELL Technologies) and fed 50 U/ml IL-2 (Thermo Fisher Scientific) every 2 to 3 d. Dynabeads were removed after 9 days of culture. T cells were seeded at 5×10⁵ cells/I mL/well in 24-well plates. Cultures were supplemented very 2 days with 50 U/mL IL-2. Every 2 or 3 days, cells were counted and sub-cultured at 5×10⁵ cells/1 mL/well in 24-well plates. Compared to the control cell, which expands over 200 times, the CAR positive cells expanded over 60,000 times (FIG. 3).

1. A polynucleotide encoding a chimeric antigen receptor (CAR), wherein the CAR comprising (1) an extracellular domain comprising a first antigen-binding domain, (2) a transmembrane domain and (3) an intracellular signaling domain, wherein the first antigen binding domain specifically binds to albumin.

2-9. (canceled)

10. The polynucleotide of claim 1, wherein the antigen-binding domain comprises the sequence of SEQ ID NO: 139, or a sequence having at least 90% identity thereto.

11. (canceled)

12. The polynucleotide of claim 10, wherein the CAR further comprises (1) signal peptide, (2) a signal peptide of CDS alpha.

13. (canceled)

14. The polynucleotide of claim 1, wherein the transmembrane domain comprises (1) a transmembrane domain of CD8 alpha, or (2) the sequence of SEQ ID NO: 133, or a sequence having at least 90% identity thereto; or a sequence having 1,2,3,4 or 5 amino acid residue difference therefrom.

15. (canceled)

16. The polynucleotide of claim 1, wherein the extracellular domain is linked to the transmembrane domain by a hinge region.

17. The polynucleotide of claim 16, wherein the hinge region comprises (1) a hinge region of CD8 alpha, or (2) the sequence of SEQ ID NO: 134, or a sequence having at least 90% identity thereto; or a sequence having 1, 2, 3, 4 or 5 amino acid residue difference therefrom.

18. (canceled)

19. The polynucleotide of claim 1, wherein the intracellular domain comprises a costimulatory domain and a signaling domain.

20. The polynucleotide of claim 19, wherein (1) the intracellular domain comprises a signaling domain of CD3 zeta; or (2) the costimulatory domain comprises an intracellular domain of CD137.

21. The polynucleotide of claim 20, wherein (1) the intracellular domain of CD137 comprises the sequence of SEQ ID NO: 135, or a sequence having at least 90% identity thereto; or a sequence having 1, 2, 3, 4 or 5 amino acid residue difference therefrom, or (2) the signaling domain of CD3 zeta comprises the sequence of SEQ ID NO: 136, or a sequence having at least 90% identity thereto.

22-23. (canceled)

24. The polynucleotide of claim 1, wherein the CAR has the structure: S-AB-H-TM-IC wherein S is a signal peptide, AB is the antigen-binding domain, H is a hinge region, TM is the transmembrane domain and IC is the intracellular signaling domain.

25. The polynucleotide of claim 24, wherein the CAR has the structure: (1)S-VH-L-VL-H-TM-IC, wherein VH is a variable heavy region, L is a linker, VL is a variable light region; (2) S-VL-L-VH-H-TM-IC, wherein VH is a variable heavy region, L is a linker, VL is a variable light region: (3) S-SDAB-H-TM-IC, wherein SDAB is a single domain antibody; or (4) S-N-H-TM-IC, wherein N is a nanobody.

26-28. (canceled)

29. The polynucleotide of claim 1, wherein the extracellular domain further comprises (1) a second antigen-binding domain specifically binding to a cancer antigen, or (2) a second antigen-binding domain specifically binding to albumin, wherein the first and second antigen-binding domains bind different epitopes of albumin.

30-32. (canceled)

33. The polynucleotide of claim 1, which is a DNA or RNA.

34. A polypeptide encoded by the polynucleotide of claim 1.

35. A vector comprising the polynucleotide of claim 1, wherein the polynucleotide encoding the CAR is operatively linked to at least one regulatory polynucleotide element for expression of the CAR.

36-37. (canceled)

38. An engineered cell comprising the polypeptide of claim 34.

39-43. (canceled)

44. A method for stimulating an immune response comprising contacting the engineered cell of claim 38 with albumin.

45-53. (canceled)

54. A method for expanding cells in vitro, the method comprising culturing the engineered cell of claim 38 in vitro in a medium comprising albumin.

55-57. (canceled)

58. A method for expanding cells in vitro, the method comprising introducing the polynucleotide of claim 1 to a cell in vitro, thereby expressing the CAR; and culturing the cell in a medium comprising albumin.

59-60. (canceled)

61. A method for treating a disease or pathological condition in a patient comprising administering a therapeutically effective amount of the engineered cell of claim 38 to the patient.

62-68. (canceled)


SEQUENCE LISTING (139 sequences)

SEQ ID NO: 1 - protein, 5 aa, synthetic construct
  REGION 1..5: note = Synthetic
NYAIN

SEQ ID NO: 2 - protein, 5 aa, synthetic construct
  REGION 1..5: note = Synthetic
NYGIH

SEQ ID NO: 3 - protein, 10 aa, synthetic construct
  REGION 1..10: note = Synthetic
GFSLSSNAMG

SEQ ID NO: 4 - protein, 11 aa, synthetic construct
  REGION 1..11: note = Synthetic
GFSFSSSYWIC

SEQ ID NO: 5 - protein, 16 aa, synthetic construct
  REGION 1..16: note = Synthetic
IIWASGTTFYATWAKG

SEQ ID NO: 6 - protein, 17 aa, synthetic construct
  REGION 1..17: note = Synthetic
SISPSGGLTYYRDSVKG

SEQ ID NO: 7 - protein, 16 aa, synthetic construct
  REGION 1..16: note = Synthetic
IISVGGFTYYASWAKG

SEQ ID NO: 8 - protein, 17 aa, synthetic construct
  REGION 1..17: note = Synthetic
CVFTGDGTTYYASWAKG

SEQ ID NO: 9 - protein, 13 aa, synthetic construct
  REGION 1..13: note = Synthetic
TVPGYSTAPYFDL

SEQ ID NO: 10 - protein, 8 aa, synthetic construct
  REGION 1..8: note = Synthetic
GGEGIFDY

SEQ ID NO: 11 - protein, 14 aa, synthetic construct
  REGION 1..14: note = Synthetic
RDRHGGDSSGAFLY

SEQ ID NO: 12 - protein, 12 aa, synthetic construct
  REGION 1..12: note = Synthetic
RPVSVYYYGMDL

SEQ ID NO: 13 - protein, 12 aa, synthetic construct
  REGION 1..12: note = Synthetic
QSSPSVWSNFLS

SEQ ID NO: 14 - protein, 12 aa, synthetic construct
  REGION 1..12: note = Synthetic
CRATQSIYNALA

SEQ ID NO: 15 - protein, 13 aa, synthetic construct
  REGION 1..13: note = Synthetic
QSSESVYSNNQLS

SEQ ID NO: 16 - protein, 11 aa, synthetic construct
  REGION 1..11: note = Synthetic
QASQIISSRSA

SEQ ID NO: 17 - protein, 7 aa, synthetic construct
  REGION 1..7: note = Synthetic
EASKLTS

SEQ ID NO: 18 - protein, 7 aa, synthetic construct
  REGION 1..7: note = Synthetic
NANTLHT

SEQ ID NO: 19 - protein, 7 aa, synthetic construct
  REGION 1..7: note = Synthetic
DASDLAS

SEQ ID NO: 20 - protein, 7 aa, synthetic construct
  REGION 1..7: note = Synthetic
QASKLAS

SEQ ID NO: 21 - protein, 11 aa, synthetic construct
  REGION 1..11: note = Synthetic
GGGYSSISDTT

SEQ ID NO: 22 - protein, 9 aa, synthetic construct
  REGION 1..9: note = Synthetic
QQYYDYPLT

SEQ ID NO: 23 - protein, 11 aa, synthetic construct
  REGION 1..11: note = Synthetic
AGGFSSSSDTA

SEQ ID NO: 24 - protein, 11 aa, synthetic construct
  REGION 1..11: note = Synthetic
QCTYIDSNFGA

SEQ ID NO: 25 - protein, 119 aa, synthetic construct
  REGION 1..119: note = Synthetic
EVQLLESGGGLVQPGGSLRLSCAVSGIDLSNYAINWVRQAPGKCLEWIGIIWASGTTFYATWAKGRFTISRDSTTVYLQMNSLRAEDTAVYYCARTVPGYSTAPYFDLWGQGTLVTVSS

SEQ ID NO: 26 - protein, 110 aa, synthetic construct
  REGION 1..110: note = Synthetic
DIVMTQSPSSVSASVGDRVTITCQSSPSVWSNFLSWYQQKPGKAPKLLIYEASKLTSGVPSRFKGSGSGTDFTLTISSLQPEDFATYYCGGGYSSISDTTFGCGTKVEIK

SEQ ID NO: 27 - protein, 121 aa, synthetic construct
  REGION 1..121: note = Synthetic
EVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 28 - protein, 112 aa, synthetic construct
  REGION 1..112: note = Synthetic
DIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLG

SEQ ID NO: 29 - protein, 234 aa, synthetic construct
  REGION 1..234: note = Synthetic
EVQLLESGGGLVQPGGSLRLSCAVSGIDLSNYAINWVRQAPGKCLEWIGIIWASGTTFYATWAKGRFTISRDSTTVYLQMNSLRAEDTAVYYCARTVPGYSTAPYFDLWGQGTLVTVSSGGGGSDIVMTQSPSSVSASVGDRVTITCQSSPSVWSNFLSWYQQKPGKAPKLLIYEASKLTSGVPSRFKGSGSGTDFTLTISSLQPEDFATYYCGGGYSSISDTTFGCGTKVEIK

SEQ ID NO: 30 - protein, 238 aa, synthetic construct
  REGION 1..238: note = Synthetic
EVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSSGGGGSDIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLG

SEQ ID NO: 31 - protein, 11 aa, synthetic construct
  REGION 1..11: note = Synthetic
RASQSIFRHLK

SEQ ID NO: 32 - protein, 11 aa, synthetic construct
  REGION 1..11: note = Synthetic
RASQSIIKHLK

SEQ ID NO: 33 - protein, 11 aa, synthetic construct
  REGION 1..11: note = Synthetic
RASQSIYYHLK

SEQ ID NO: 34 - protein, 11 aa, synthetic construct
  REGION 1..11: note = Synthetic
RASQYIGRYLR

SEQ ID NO: 35 - protein, 11 aa, synthetic construct
  REGION 1..11: note = Synthetic
RASQWIGRYLR

SEQ ID NO: 36 - protein, 11 aa, synthetic construct
  REGION 1..11: note = Synthetic
RASQYISRQLR

SEQ ID NO: 37 - protein, 11 aa, synthetic construct
  REGION 1..11: note = Synthetic
RASQWIHRQLK

SEQ ID NO: 38 - protein, 11 aa, synthetic construct
  REGION 1..11: note = Synthetic
RASQHIHRELR

SEQ ID NO: 39 - protein, 11 aa, synthetic construct
  REGION 1..11: note = Synthetic
RASQSIGRRLK

SEQ ID NO: 40 - protein, 11 aa, synthetic construct
  REGION 1..11: note = Synthetic
RASQKIYKNLR

SEQ ID NO: 41 - protein, 11 aa, synthetic construct
  REGION 1..11: note = Synthetic
RASQKIYNNLR

SEQ ID NO: 42 - protein, 11 aa, synthetic construct
  REGION 1..11: note = Synthetic
RASQWIYKSLG

SEQ ID NO: 43 - protein, 11 aa, synthetic construct
  REGION 1..11: note = Synthetic
RASQWIYRHLR

SEQ ID NO: 44 - protein, 11 aa, synthetic construct
  REGION 1..11: note = Synthetic
RASQSISSYLN

SEQ ID NO: 45 - protein, 11 aa, synthetic construct
  REGION 1..11: note = Synthetic
RASQKIATYLN

SEQ ID NO: 46 - protein, 5 aa, synthetic construct
  REGION 1..5: note = Synthetic
PYTMS

SEQ ID NO: 47 - protein, 5 aa, synthetic construct
  REGION 1..5: note = Synthetic
AYQMA

SEQ ID NO: 48 - protein, 5 aa, synthetic construct
  REGION 1..5: note = Synthetic
DYDMT

SEQ ID NO: 49 - protein, 5 aa, synthetic construct
  REGION 1..5: note = Synthetic
DYVMG

SEQ ID NO: 50 - protein, 5 aa, synthetic construct
  REGION 1..5: note = Synthetic
HYRMG

SEQ ID NO: 51 - protein, 5 aa, synthetic construct
  REGION 1..5: note = Synthetic
WDKMG

SEQ ID NO: 52 - protein, 5 aa, synthetic construct
  REGION 1..5: note = Synthetic
AYPMS

SEQ ID NO: 53 - protein, 5 aa, synthetic construct
  REGION 1..5: note = Synthetic
TYTMA

SEQ ID NO: 54 - protein, 5 aa, synthetic construct
  REGION 1..5: note = Synthetic
PTNMS

SEQ ID NO: 55 - protein, 7 aa, synthetic construct
  REGION 1..7: note = Synthetic
AASRLQS

SEQ ID NO: 56 - protein, 7 aa, synthetic construct
  REGION 1..7: note = Synthetic
GASRLQS

SEQ ID NO: 57 - protein, 7 aa, synthetic construct
  REGION 1..7: note = Synthetic
KASTLQS

SEQ ID NO: 58 - protein, 7 aa, synthetic construct
  REGION 1..7: note = Synthetic
DSSVLQS

SEQ ID NO: 59 - protein, 7 aa, synthetic construct
  REGION 1..7: note = Synthetic
NGSQLQS

SEQ ID NO: 60 - protein, 7 aa, synthetic construct
  REGION 1..7: note = Synthetic
GAVSLQS

SEQ ID NO: 61 - protein, 7 aa, synthetic construct
  REGION 1..7: note = Synthetic
YASILQS

SEQ ID NO: 62 - protein, 7 aa, synthetic construct
  REGION 1..7: note = Synthetic
QASRLQS

SEQ ID NO: 63 - protein, 7 aa, synthetic construct
  REGION 1..7: note = Synthetic
RTSWLQS

SEQ ID NO: 64 - protein, 7 aa, synthetic construct
  REGION 1..7: note = Synthetic
NSSILQS

SEQ ID NO: 65 - protein, 7 aa, synthetic construct
  REGION 1..7: note = Synthetic
NTSILQS

SEQ ID NO: 66 - protein, 7 aa, synthetic construct
  REGION 1..7: note = Synthetic
QSSLLQS

SEQ ID NO: 67 - protein, 7 aa, synthetic construct
  REGION 1..7: note = Synthetic
DASRLQS

SEQ ID NO: 68 - protein, 7 aa, synthetic construct
  REGION 1..7: note = Synthetic
RNSFLQS

SEQ ID NO: 69 - protein, 7 aa, synthetic construct
  REGION 1..7: note = Synthetic
RSSSLQS

SEQ ID NO: 70 - protein, 7 aa, synthetic construct
  REGION 1..7: note = Synthetic
RLSVLQS

SEQ ID NO: 71 - protein, 7 aa, synthetic construct
  REGION 1..7: note = Synthetic
RNSQLQS

SEQ ID NO: 72 - protein, 7 aa, synthetic construct
  REGION 1..7: note = Synthetic
RNSPLQS

SEQ ID NO: 73 - protein, 17 aa, synthetic construct
  REGION 1..17: note = Synthetic
TISPFGSTTYYADSVKG

SEQ ID NO: 74 - protein, 17 aa, synthetic construct
  REGION 1..17: note = Synthetic
TIHQTGFSTYYADSVKG

SEQ ID NO: 75 - protein, 17 aa, synthetic construct
  REGION 1..17: note = Synthetic
MISSSGLWTYYADSVKG

SEQ ID NO: 76 - protein, 17 aa, synthetic construct
  REGION 1..17: note = Synthetic
LIKPNGSPTYYADSVKG

SEQ ID NO: 77 - protein, 17 aa, synthetic construct
  REGION 1..17: note = Synthetic
WIRPDGTFTYYADSVKG

SEQ ID NO: 78 - protein, 17 aa, synthetic construct
  REGION 1..17: note = Synthetic
FIGREGYGTYYADSVKG

SEQ ID NO: 79 - protein, 17 aa, synthetic construct
  REGION 1..17: note = Synthetic
SISSWGTGTYYADSVKG

SEQ ID NO: 80 - protein, 16 aa, synthetic construct
  REGION 1..16: note = Synthetic
SITSSGSTYYADSVKG

SEQ ID NO: 81 - protein, 17 aa, synthetic construct
  REGION 1..17: note = Synthetic
TITGTGAATYYADSVKG

SEQ ID NO: 82 - protein, 9 aa, synthetic construct
  REGION 1..9: note = Synthetic
QQVALYPKT

SEQ ID NO: 83 - protein, 9 aa, synthetic construct
  REGION 1..9: note = Synthetic
QQGARWPQT

SEQ ID NO: 84 - protein, 9 aa, synthetic construct
  REGION 1..9: note = Synthetic
QQVRKVPQT

SEQ ID NO: 85 - protein, 9 aa, synthetic construct
  REGION 1..9: note = Synthetic
QQRYRMPYT

SEQ ID NO: 86 - protein, 9 aa, synthetic construct
  REGION 1..9: note = Synthetic
QQRYMQPFT

SEQ ID NO: 87 - protein, 9 aa, synthetic construct
  REGION 1..9: note = Synthetic
QQRYLQPYT

SEQ ID NO: 88 - protein, 9 aa, synthetic construct
  REGION 1..9: note = Synthetic
QQRYITYNT

SEQ ID NO: 89 - protein, 9 aa, synthetic construct
  REGION 1..9: note = Synthetic
QQRYSSPYT

SEQ ID NO: 90 - protein, 9 aa, synthetic construct
  REGION 1..9: note = Synthetic
QQTFSKPST

SEQ ID NO: 91 - protein, 9 aa, synthetic construct
  REGION 1..9: note = Synthetic
QQKYLPPYT

SEQ ID NO: 92 - protein, 9 aa, synthetic construct
  REGION 1..9: note = Synthetic
QQRYRVPYT

SEQ ID NO: 93 - protein, 9 aa, synthetic construct
  REGION 1..9: note = Synthetic
QQTSQWPHT

SEQ ID NO: 94 - protein, 9 aa, synthetic construct
  REGION 1..9: note = Synthetic
QQRYLSPYT

SEQ ID NO: 95 - protein, 9 aa, synthetic construct
  REGION 1..9: note = Synthetic
QQRWRAPYT

SEQ ID NO: 96 - protein, 9 aa, synthetic construct
  REGION 1..9: note = Synthetic
QQYHQMPRT

SEQ ID NO: 97 - protein, 9 aa, synthetic construct
  REGION 1..9: note = Synthetic
QQTHNPPKT

SEQ ID NO: 98 - protein, 9 aa, synthetic construct
  REGION 1..9: note = Synthetic
QQTYTVPPT

SEQ ID NO: 99 - protein, 9 aa, synthetic construct
  REGION 1..9: note = Synthetic
QQTYAVPPT

SEQ ID NO: 100 - protein, 9 aa, synthetic construct
  REGION 1..9: note = Synthetic
QQTYNVPPT

SEQ ID NO: 101 - protein, 9 aa, synthetic construct
  REGION 1..9: note = Synthetic
QQTFAVPPT

SEQ ID NO: 102 - protein, 9 aa, synthetic construct
  REGION 1..9: note = Synthetic
QQTYRLPPT

SEQ ID NO: 103 - protein, 7 aa, synthetic construct
  REGION 1..7: note = Synthetic
GGKDFDY

SEQ ID NO: 104 - protein, 8 aa, synthetic construct
  REGION 1..8: note = Synthetic
GNLEPFDY

SEQ ID NO: 105 - protein, 8 aa, synthetic construct
  REGION 1..8: note = Synthetic
KLSNGFDY

SEQ ID NO: 106 - protein, 9 aa, synthetic construct
  REGION 1..9: note = Synthetic
VVKDNTFDY

SEQ ID NO: 107 - protein, 9 aa, synthetic construct
  REGION 1..9: note = Synthetic
NTGGKQFDY

SEQ ID NO: 108 - protein, 9 aa, synthetic construct
  REGION 1..9: note = Synthetic
KTGPSSFDY

SEQ ID NO: 109 - protein, 11 aa, synthetic construct
  REGION 1..11: note = Synthetic
RTENRGVSFDY

SEQ ID NO: 110 - protein, 13 aa, synthetic construct
  REGION 1..13: note = Synthetic
SDVLKTGLDGFDY

SEQ ID NO: 111 - protein, 11 aa, synthetic construct
  REGION 1..11: note = Synthetic
VRSMRPYKFDY

SEQ ID NO: 112 - protein, 11 aa, synthetic construct
  REGION 1..11: note = Synthetic
GFRLFPRTFDY

SEQ ID NO: 113 - protein, 12 aa, synthetic construct
  REGION 1..12: note = Synthetic
GRGRFNVLQFDY

SEQ ID NO: 114 - protein, 9 aa, synthetic construct
  REGION 1..9: note = Synthetic
SYMGDRFDY

SEQ ID NO: 115 - protein, 7 aa, synthetic construct
  REGION 1..7: note = Synthetic
SVASFDY

SEQ ID NO: 116 - protein, 8 aa, synthetic construct
  REGION 1..8: note = Synthetic
GGQGSFDY

SEQ ID NO: 117 - protein, 9 aa, synthetic construct
  REGION 1..9: note = Synthetic
VNSLYKFDY

SEQ ID NO: 118 - protein, 9 aa, synthetic construct
  REGION 1..9: note = Synthetic
QNSRYRFDY

SEQ ID NO: 119 - protein, 117 aa, synthetic construct
  REGION 1..117: note = Synthetic
EVQLLESGGGLVQPGGSLRLSCAASGFTFRTYTMAWVRQAPGKGLEWVSSITSSGSTYYADSVKGRFTISRDNSKNTLYLQMNSLRAEDTAVYYCAKVNSLYKFDYWGQGTLVTVSS

SEQ ID NO: 120 - protein, 118 aa, synthetic construct
  REGION 1..118: note = Synthetic
EVQLLESGGGLVQPGGSLRLSCAASGFTFRPTNMSWVRQAPGKGLEWVSTITGTGAATYYADSVKGRFTISRDNSKNTLYLQMNSLRAEDTAVYYCAKQNSRYRFDYWGQGTLVTVSS

SEQ ID NO: 121 - protein, 108 aa, synthetic construct
  REGION 1..108: note = Synthetic
DIQMTQSPSSLSASVGDRVTITCRASQSIFRHLKWYQQKPGKAPKLLIYAASRLQSGVPSRFSGSGSGTDFTLTISSLQPEDFATYYCQQVALYPKTFGQGTKVEIKR

SEQ ID NO: 122 - protein, 108 aa, synthetic construct
  REGION 1..108: note = Synthetic
DIQMTQSPSSLSASVGDRVTITCRASQSISSYLNWYQQKPGKAPKLLIYRNSPLQSGVPSRFSGSGSGTDFTLTISSLQPEDFATYYCQQTYRVPPTFGQGTKVEIKR

SEQ ID NO: 123 - protein, 5 aa, synthetic construct
  REGION 1..5: note = Synthetic
TTCMA

SEQ ID NO: 124 - protein, 5 aa, synthetic construct
  REGION 1..5: note = Synthetic
DYTTG

SEQ ID NO: 125 - protein, 17 aa, synthetic construct
  REGION 1..17: note = Synthetic
TITTGGTYPYYADSVLG

SEQ ID NO: 126 - protein, 16 aa, synthetic construct
  REGION 1..16: note = Synthetic
CISRSDGNTYYAESVL

SEQ ID NO: 127 - protein, 18 aa, synthetic construct
  REGION 1..18: note = Synthetic
DASWGCRLSGSWSTVYNY

SEQ ID NO: 128 - protein, 16 aa, synthetic construct
  REGION 1..16: note = Synthetic
ADRYRSGFLGNGYEYD

SEQ ID NO: 129 - protein, 127 aa, synthetic construct
  REGION 1..127: note = Synthetic
QVQLQGSGGGSVQAGGSLRLSCAASAYTYSTTCMAWFRQAPGKEREKVATITTGGTYPYYADSVKGRFTISQDNAKNTVYLQMNSLKPEDTAMYYCAADASWGCRLSGSWSTVYNYWGQGTQVTVSS

SEQ ID NO: 130 - protein, 123 aa, synthetic construct
  REGION 1..123: note = Synthetic
DVQLQASGGDLVQAGGSLRLSCHASGLDDYIIGWFRQAPGKEREGLSCISRSDGNTYYAESVKGRFTISSDNAKNTVYLQMDSLKPEDTAVYYCAADRYRSGFLGNGYEYDYWGQGTQVTVSS

SEQ ID NO: 131 - protein, 21 aa, synthetic construct
  REGION 1..21: note = Synthetic
MALPVTALLLPLALLLHAARP

SEQ ID NO: 132 - protein, 108 aa, synthetic construct; identical to SEQ ID NO: 121
  REGION 1..108: note = Synthetic

SEQ ID NO: 133 - protein, 21 aa, synthetic construct
  REGION 1..21: note = Synthetic
IYIWAPLAGTCGVLLLSLVIT

SEQ ID NO: 134 - protein, 45 aa, synthetic construct
  REGION 1..45: note = Synthetic
TTTPAPRPPTPAPTIASQPLSLRPEACRPAAGGAVHTRGLDFACD

SEQ ID NO: 135 - protein, 42 aa, synthetic construct
  REGION 1..42: note = Synthetic
KRGRKKLLYIFKQPFMRPVQTTQEEDGCSCRFPEEEEGGCEL

SEQ ID NO: 136 - protein, 112 aa, synthetic construct
  REGION 1..112: note = Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 15 aa, synthetic construct
  REGION 1..15: note = Synthetic
GGGGSGGGGSGGGGS

SEQ ID NO: 138 - protein, 363 aa, synthetic construct
MALPVTALLLPLALLLHAARPEVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKSDVLKTGLDGFDYWGQGTLVTVSSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 122 aa, synthetic construct
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKSDVLKTGLDGFDYWGQGTLVTVSS